(19) United States
(12) Patent Application Publication
(10) Pub. No.: US 2012/0329837 A1
(43) Pub. Date: Dec. 27, 2012

(54) CATHEPSIN INHIBITORS FOR TREATING MICROGLIA-MEDIATED NEURON LOSS IN THE CENTRAL NERVOUS SYSTEM

(75) Inventor: Robert Booth, Los Altos Hills, CA (US)

(73) Assignee: ViroBay, Inc., Menlo Park, CA (US)

(21) Appl. No.: 13/525,845

(22) Filed: Jun. 18, 2012

Related U.S. Application Data
(60) Provisional application No. 61/498,486, filed on Jun. 17, 2011.

(51) Int. Cl. A61K 31/277; A61P 25/00; A61P 25/16; A61K 31/4406; A61P 25/28

(52) U.S. Cl. 514/357; 514/521

(57) ABSTRACT

The present invention concerns methods of using Cathepsin S inhibitors and compounds of Formula I that are inhibitors of cathepsin S in treating CNS disorders, diseases, and injuries, particularly neurodegenerative conditions. The present invention is directed to pharmaceutical compositions comprising these compounds for treating CNS disorders.

### CROSS-REFERENCES TO RELATED APPLICATIONS

This application claims priority to U.S. Provisional Patent Application No. 61/498,486 filed Jun. 17, 2011, the disclosure of which is incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE INVENTION

The present invention is directed to compounds that are inhibitors of cysteine S and have favorable brain penetration.

### BACKGROUND OF THE INVENTION

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increased expression or enhanced activation, however, may have pathological consequences. In this regard, certain cysteine proteases are associated with a number of disease states, including, inflammation, tumor invasion, glomerulonephritis, malaria, periodontal disease, metachromatic leukodystrophy and others. For example, increased cathepsin B levels and redistribution of the enzyme are found in tumors; thus, suggesting a role for the enzyme in tumor invasion and metastasis. In addition, cathepsin B activity is implicated in such disease states as rheumatoid arthritis, osteoarthritis, pneumocystis carinii, acute pancreatitis, inflammatory airway disease and bone and joint disorders.

Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis, neuropathic pain, and Hashimoto's thyroiditis.

Microglia, which function as immune cells in the brain, can phagocytose amyloid-β and participate in brain inflammatory processes. Recently two-photon in vivo imaging of neuron loss in the intact brain of living Alzheimer's disease model mice indicate microglia are involved in neuron elimination, as evidenced by an increased number and migration velocity of microglia locally around lost neurons. Knockout of the fractalkine/CX3CL1 receptor, CX3CR1, which is critical in neuron-microglia communication, prevented neuron loss (see, Fuhrmann et al., Nature Neuroscience 13(4):411-413 (2010)). CX3CR1 deficiency alters microglial activation and reduces beta-amyloid deposition in two Alzheimer's disease mouse models (see, Lee et al., Am J Pathol. 177(5):2549-62 (2010)). CatS mRNA in brain has been found to be expressed in microglia, where the enzyme is essential for antigen presentation and turnover of intracellular and extracellular proteins in tissue remodeling (Petanceska S, Canoll P, Devi L A, J Biol Chem 271:4403-4409, 7 (1996); Riese R J, et al., Immunity 4:357-366 (1996)). Soluble fractalkine CX3CL1 is released from its membrane bound form by cathepsin S in vivo in the CNS during neuronal injury and this activation and neuronal-glial communication is disrupted by intrathecal administration of Cat S inhibitors. (see, Clark et al., Proc Natl Acad Sci USA. 104(25):10655-60 (2007) and Clark et al., J. Neurosci. 27; 29(21):6945-54 (2009)).

In view of the number of CNS inflammatory diseases associated with microglia-mediated inflammation and neuron loss, methods of treating such diseases are needed. This invention provides for these and other needs by providing methods of treating such diseases by administering Cat S inhibitors which are centrally active upon systemic administration.

### BRIEF SUMMARY OF THE INVENTION

In its various aspects, the invention provides methods for reducing microglia-mediated neuro-inflammation and preventing or reducing microglia-mediated neuronal loss in CNS disorders by systemically administering a therapeutically active amount of a centrally active cathepsin S inhibitor to a subject in need thereof. In some embodiments, subject has a CNS disorder associated with neuronal loss mediated by microglial cells. In some embodiments, the disease is Parkinson's disease, Alzheimer's disease, post operative cognitive dysfunction, dementia, traumatic brain injury, amyotrophic lateral sclerosis, and stroke. In any of the above embodiments, the cathepsin S inhibitor for use according to the invention is a compound of the Formula (I):

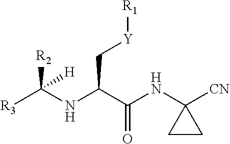

in which Y is SO₂ or CF₂, R₁ is alkyl, cycloalkyl, alkylcycloalkyl, aryl, aralkyl, or pyridinylalkyl; R₂ is CHF₂, CF₃, C₂F₅, or CF₂Oaryl; R₃ is H or aryl, wherein any aryl rings can be optionally substituted with 1 or 2 substituents selected from halo, lower alkyl, CF₃, lower alkoxy, or OCF₃.

In a preferred embodiment, Y is SO₂. In a particularly preferred embodiment of such, the compound of Formula I is selected from the group consisting of

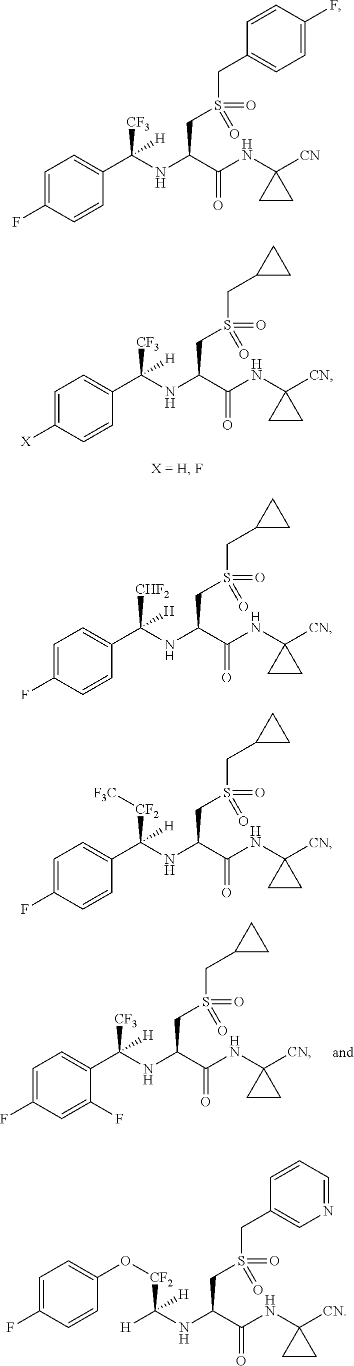

In another set of preferred embodiments, Y is CF₂ and R₂ is CF₂Oaryl. In another embodiment of such, the compound is

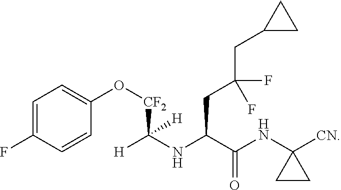

In another set of embodiments, Y is CF₂. In further of such embodiments, the compound of Formula I is selected from the group consisting of:

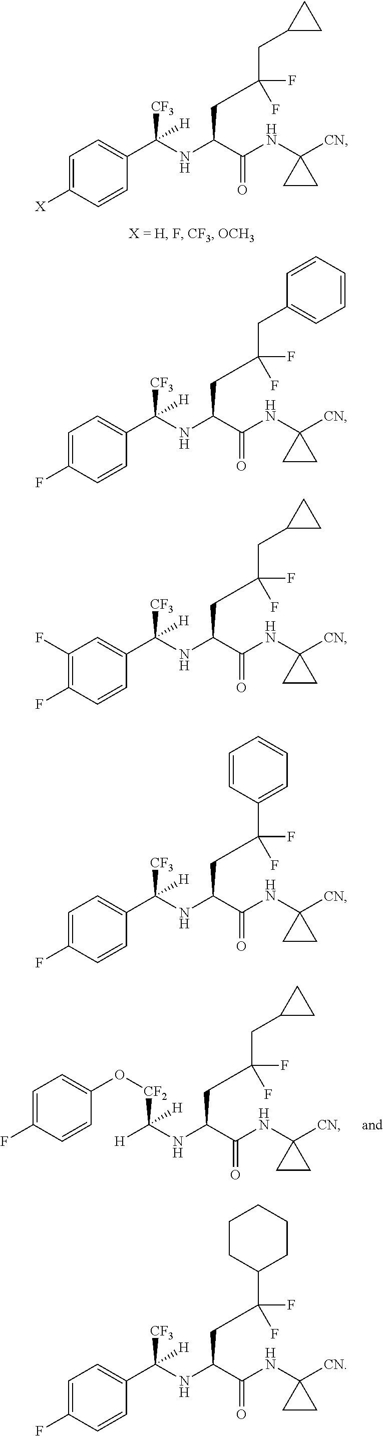

In another set of embodiments, Y is CF₂. In further of such embodiments, the compound of Formula I is selected from the group consisting of:

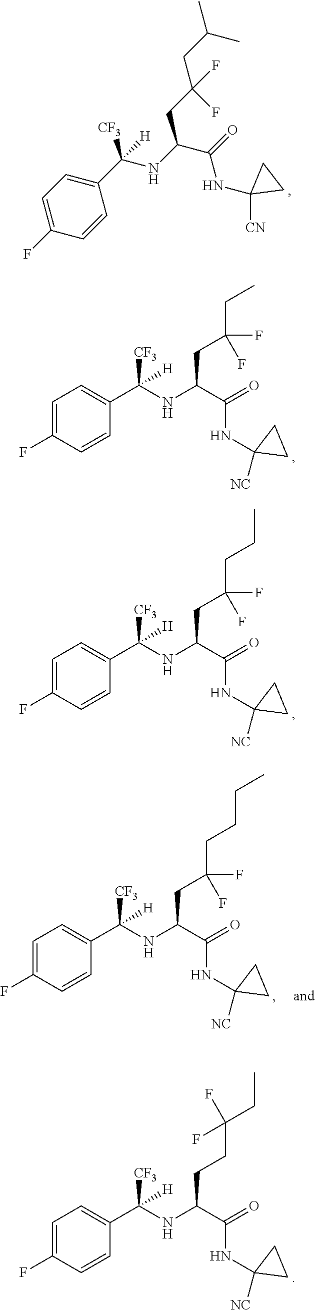

In another set of embodiments, Y is CF₂. In further of such embodiments, the compound of Formula I is selected from the group consisting of:

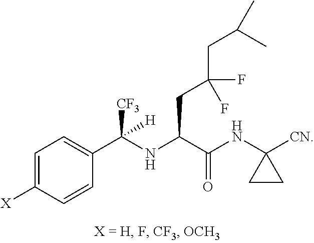

In another set of embodiments, Y is SO₂. In further of such embodiments, the compound of Formula I is selected from the group consisting of:

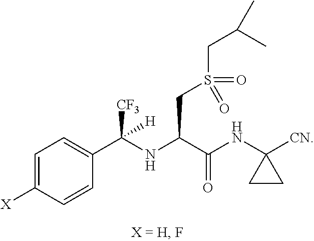

In other embodiments of any of the above, the aryl member of R₃ is substituted with F, CF₃, or OCH₃. In still further embodiments of such, the R₃ aryl member is a 4-fluoro, 4-methoxy, or 4-trifluoromethoxyphenyl member.

### BRIEF DESCRIPTION OF THE DRAWINGS

FIG. 1. The amounts of cathepsin S inhibitors in rat and mouse brain. Cathepsin S inhibitors, Compounds A, B, C and D, were each formulated in a methylcellulose/Tween 80 suspension and dosed by oral gavage with a single dose in mice. At the indicated time 3 hours after dosing, samples were harvested and frozen for pharmacokinetic assessment of drug concentrations in the plasma, brain tissue, and blood-free cerebral spinal fluid (CSF). Bioanalytical analysis of these samples was conducted to determine the concentration of each compound in each compartment. For the calculations of drug concentration in brain tissue, it was assumed that the density of wet brain tissue is 1 g/mL. 6 mice were dosed in each group with each compound, and the average plasma, brain, and CSF drug concentrations in all 6 mice was assessed.

FIG. 2. Structures and human cathepsin S Kᵢ values for the compounds A, B, C and D of FIG. 1 are provided. Cathepsin S assay performed as described in examples.

### DETAILED DESCRIPTION OF THE INVENTION

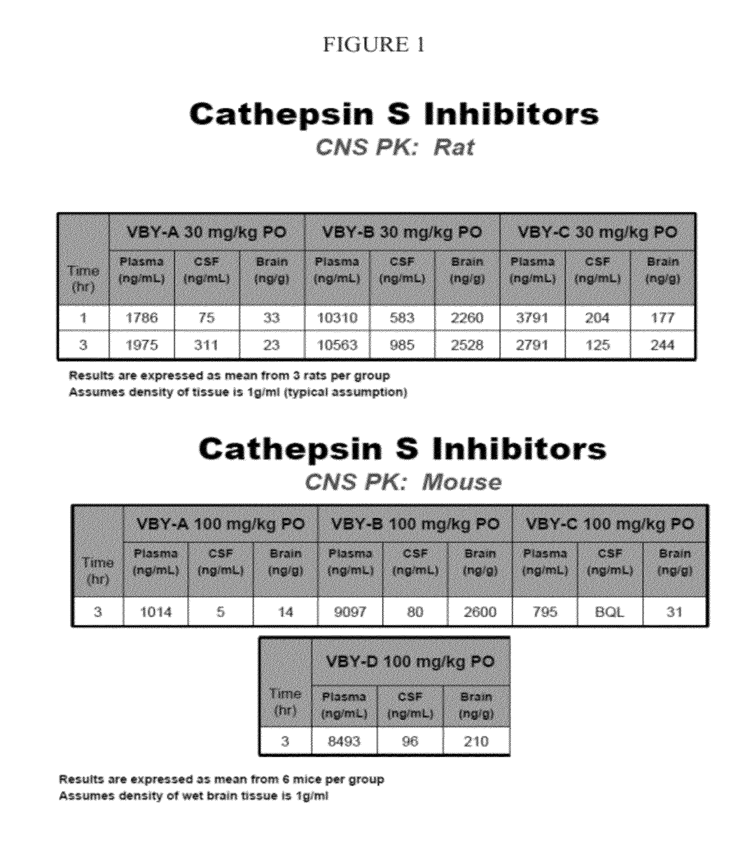

The loss of neurons associated with both Alzheimer's disease and brain injuries can be mediated by microglia and modulated by the microglial fractalkine receptor CX3CR1. Soluble fractalkine (CX3CL1) is released from its membrane-bound form within the CNS by cathepsin S during neuronal injury and cathepsin S inhibitors can inhibit the release of free fractalkine and the subsequent activation of the fractalkine receptor (CX3CR1) which is important for the recruitment and activation of the microglia which precedes the elimination of neurons. The present invention relates to the discovery that nitrile-containing cathepsin inhibitors of Formula I have a greatly superior ability to enter brain tissue following systemic administration than other cathepsin S inhibitors. The nitrile compounds are particularly superior in this regard as compared to ketoamide cathepsin S inhibitors. Evidence for this highly superior ability to enter the CNS is shown by the much greater levels of VBY-B (2600 ng/g) and VBY-D (210 ng/g) in mouse brain as compared to VBY-A (14 ng/g) or VBY-C (31 ng/g). For VBY-B, these differences were about 185-fold and 84-fold, respectively (FIG. 1). For the rat, VBY-B was about 104-fold superior over VBY-A and about 10-fold superior over VBY-C. Accordingly, this invention provides methods of treating CNS disorders in which cathepsin S and/or soluble fractalkine (CX3CL1) activity contributes to the pathophysiology of the disorder by systemically administering to the subject a therapeutically effective amount of a cathepsin S inhibitor of Formula I.

Accordingly, in some embodiments, the invention provides methods for preventing or reducing neuron loss mediated by microglia in the central nervous system, said method comprising systemically administering to a subject in need thereof a therapeutically effective amount of a cathepsin S inhibitor of Formula I:

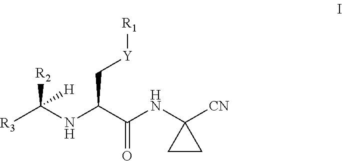

In some embodiments of the cathepsin S inhibitors of Formula I, Y is SO₂ or CF₂. In certain embodiments, Y is SO₂. In some other embodiments, Y is CF₂. In certain embodiments, R₁ is alkyl, cycloalkyl, alkylcycloalkyl, aryl, aralkyl, or pyridinylalkyl. In yet other embodiments, R₁ is cycloalkyl, alkylcycloalkyl, aryl, aralkyl, or pyridinylalkyl. In still other embodiments, R₂ is CHF₂, CF₃, C₂F₅, or CF₂Oaryl. In other embodiments, R₃ is H or aryl.

In some embodiments, wherein: Y is SO₂ or CF₂, R₁ is alkyl, cycloalkyl, alkylcycloalkyl, aryl, aralkyl, or pyridinylalkyl; R₂ is CHF₂, CF₃, C₂F₅, or CF₂Oaryl; and R₃ is H or aryl, any aryl members can be optionally substituted with 1 or 2 substituents selected from halo, lower alkyl, CF₃, lower alkoxy, and OCF₃. In a preferred embodiment, R₂ is CF₂Oaryl when Y is CF₂. In another preferred embodiment, Y is SO₂. In yet another embodiment, Y is CF₂.

In some embodiments, the invention provides cathepsin S inhibitors of Formula I wherein R₁ is alkyl, cycloalkyl, alkylcycloalkyl, aryl, aralkyl, or pyridinylalkyl optionally substituted with 1 or 2 substituents selected from halo, lower alkyl, CF₃, lower alkoxy, or OCF₃. In other embodiments, R₁ is phenyl, benzyl, 4-fluoro-phenyl, 4-fluoro-phenyl-methyl, cyclopropyl, cyclopropylmethyl, pyridinyl, or pyridinyl-methyl. In certain of these embodiments, Y is SO₂. In other embodiments, Y is CF₂. In some embodiments, when Y is SO₂, R₁ is pyridinylmethyl.

In other embodiments, Y—R₁ is

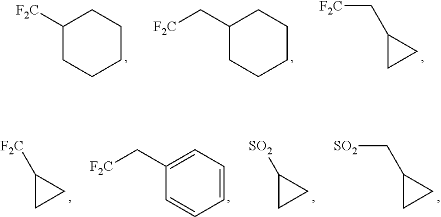

pyridinyl-methyl-sulfonyl, or oxopyridinyl-methyl-sulfonyl.

In certain embodiments, R₂ is CF₃, CF₂—CF₃, or

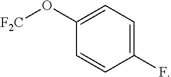

In some embodiments, R₂ is CHF₂. In some other embodiments, R₂ is CF₂CF₃. In some other embodiments, R₂ is CF₃.

In some embodiments, R₃ is 4-fluoro-phenyl, phenyl, 4-methoxy-phenyl, 2,4-difluorophenyl, or 3,4-difluorophenyl.

In other embodiments, of any of the above, the neuronal loss is due to Parkinson's disease, Alzheimer's disease, post operative cognitive dysfunction, or dementia. In yet other embodiments of any of the above, the neuronal loss is due to a neurodegenerative condition. In still other embodiments, the disease is due to a traumatic brain injury, concussion, exposure to a neurotoxin, cerebral hypoxia, cerebral stroke or by inadequate brain perfusion.

Accordingly, the invention also provides methods of treating a disease selected from Parkinson's disease, Alzheimer's disease, post operative cognitive dysfunction, dementia, stroke, or concussion by systemically administering to a human subject in need thereof a therapeutically effective amount of a compound of Formula I.

In addition the invention provides methods of treating a brain injury, including neurotoxic injuries and injuries due to a traumatic brain injury, hypoxia, or concussion, said method comprising systemically administering to a subject in need thereof a therapeutically effective amount of a compound of Formula I.

### DEFINITIONS

Unless otherwise stated, the following terms used in the specification and claims are defined for the purposes of this Application and have the following meanings.

“Neurodegenerative diseases or disorders” refers to hereditary and sporadic conditions which are characterized by progressive nervous system dysfunction and neuron loss. These disorders are often associated with atrophy of the affected central or peripheral nervous system structures. Alzheimer's disease, Parkinson's disease, Creutzfeldt-Jakob, as well as multiple sclerosis, are due to neuronal degeneration in the central nervous system. Exemplary neurodegenerative diseases, include but are not limited to age-related cognitive decline, early Alzheimer's disease as seen in Mild Cognitive Impairment (“MCI”), vascular dementia, or Alzheimer's disease, which can be sporadic (non-hereditary) Alzheimer's disease or familial (hereditary) Alzheimer's disease. The neurodegenerative diseases can also be cerebral amyloid angiopathy or hereditary cerebral hemorrhage, senile dementia, Down's syndrome, or inclusion body myositis. Other neurodegenerative diseases or disorders include but are not limited to those involving Lewy bodies, such as dementia with Lewy bodies, multiple system atrophy, and Hallervorden-Spatz disease.

“Amyloidosis” or “amyloid disease” refers to a pathological condition characterized by the presence of increased amyloid fibers in the CNS and neuron loss. Local deposits of amyloid is particularly found in elderly individuals. The most frequent type of amyloid in the brain is composed primarily of β-amyloid fibrils and is associated with dementia associated with both sporadic (non-hereditary) and hereditary Alzheimer's disease. Amyloid disease includes Down's syndrome, cerebral amyloid angiopathy, inclusion body myositis (“IBM”), age-related macular degeneration and senile dementia.

“Traumatic brain injury” or “TBI” is a brain injury due to physical forces related to direct impact (coup and counter coup injuries, shock waves, or acceleration). Even in the absence of an impact, significant acceleration or deceleration of the head can cause TBI; however in most cases a combination of impact and acceleration is present. Forces from the head striking or being struck by something, termed contact or impact loading typically cause focal injuries, and movement of the brain within the skull, termed noncontact or inertial loading, usually causes diffuse injuries.

Alzheimer's disease (AD), the most prevalent neurodegenerative disease, is characterized clinically by progressive memory loss and cognitive dysfunction, and pathologically by the development in the brain of intracellular neurofibrillary tangles containing abnormally hyperphosphorylated tau and extracellular senile amyloid plaques constituted predominantly of β-amyloid. AD is associated with several genetic traits, each related to metabolism of the amyloid precursor protein (APP) and its cleavage to form β-amyloid peptides. Yet, the more common form(s) of AD do not appear to have a single genetic cause. All forms of AD appear to share in common increased production or accumulation of β-amyloid peptides, especially Amyloid β42. Microglial activation appears to mediate much of the neuronal loss associated with AD (see, Fuhrmann et al., Nature Neuroscience 13(4):411-413 (2010)).

“Parkinson's disease”, also referred to as “PD”, is a chronic disease of the central nervous system affecting voluntary movement. Parkinson's disease is characterized by the presence of Lewy bodies and the loss of dopamine-producing neurons in substantia nigra that controls muscle movement. The Lewy body contains a protein called α-synuclein, which plays the central role in Parkinson's disease and other diseases involving Lewy bodies, such as dementia with Lewy bodies, multiple system atrophy, and Hallervorden-Spatz disease (Jellinger, Mov Disord. 18 (Suppl 6): S2-12, 2003).

“Therapeutically effective amount” means that amount which, when administered to an animal for treating a disease, is sufficient to effect such treatment for the disease.

“Treatment” or “treating” means any administration of a compound of the present invention and includes:
(1) preventing or delaying the disease or condition from occurring in an animal which may be predisposed to the disease but does not yet experience or display the pathology or symptomatology of the disease,
(2) inhibiting the disease or condition in an animal that is experiencing or displaying the pathology or symptomatology of the disease (i.e., arresting further development of the pathology and/or symptomatology), or
(3) ameliorating the disease or condition in an animal that is experiencing or displaying the pathology or symptomatology of the diseased (i.e., reversing the pathology and/or symptomatology).

“Pathology” of a disease means the essential nature, causes and development of the disease as well as the structural and functional changes that result from the disease processes.

“Animal” includes humans, non-human mammals (e.g., dogs, cats, rabbits, cattle, horses, sheep, goats, swine, deer, and the like) and non-mammals (e.g., birds, and the like).

In some embodiments, the subject is a human having a condition selected from a neurodegenerative disease or condition, Parkinson's disease, amyloidosis, dementia, a traumatic brain injury or concussion, neurodegenerative diseases, include but are not limited to, early Alzheimer's disease as seen in Mild Cognitive Impairment (“MCI”), vascular dementia, or Alzheimer's disease, which can be sporadic (non-hereditary) Alzheimer's disease or familial (hereditary) Alzheimer's disease. The neurodegenerative diseases can also be cerebral amyloid angiopathy or hereditary cerebral hemorrhage, senile dementia, Down's syndrome, inclusion body myositis, or age-related macular degeneration. Other neurodegenerative diseases or disorders include but are not limited to those involving Lewy bodies, such as dementia with Lewy bodies, multiple system atrophy, and Hallervorden-Spatz disease.

“Aryl” refers to a monocyclic or fused bicyclic ring assembly containing 6, 7, 8, 9 or 10 ring carbon atoms wherein each ring is aromatic e.g., phenyl or naphthyl. “Aralkyl” refers to an -(alkylene)-R radical where R is aryl as defined above e.g., benzyl, phenethyl, and the like. “Aryloxy” refers to an —OR radical where R is aryl as defined above e.g., phenoxy, and the like.

“Alkyl” represented by itself means a straight or branched, saturated aliphatic radical containing one to eight carbon atoms, unless otherwise indicated e.g., alkyl includes methyl, ethyl, propyl, isopropyl, butyl, sec-butyl, isobutyl, tert-butyl, and the like.

“Alkylene”, unless indicated otherwise, means a straight or branched, saturated aliphatic, divalent radical having the number of one to six carbon atoms, e.g., methylene (—CH₂—), ethylene (—CH₂CH₂—), trimethylene (—CH₂CH₂CH₂—), tetramethylene (—CH₂CH₂CH₂CH₂—) 2-methyltetramethylene (—CH₂CH(CH₃)CH₂CH₂—), pentamethylene (—CH₂CH₂CH₂CH₂CH₂—), and the like.

“Alkoxy” refers to a —OR radical where R is an alkyl group as defined above e.g., methoxy, ethoxy, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxy-ethyl, 1-, 2-, or 3-methoxypropyl, 2-ethoxyethyl, and the like.

“Aromatic” refers to a moiety wherein the constituent atoms make up an unsaturated ring system, all atoms in the ring system are sp² hybridized and the total number of pi electrons is equal to 4 n+2.

“Cycloalkyl” refers to a monovalent saturated monocyclic ring containing three to eight ring carbon atoms e.g., cyclopropyl, cyclobutyl, cyclopentyl, cyclohexyl, and the like.

“Cycloalkylalkyl” refers to a -(alkylene)-R radical where R is cycloalkyl as defined above e.g., cyclopropylmethyl, cyclobutylethyl, cyclobutylmethyl, and the like.

“Disease” specifically includes any unhealthy condition of an animal or part thereof and includes an unhealthy condition that may be caused by, or incident to, medical or veterinary therapy applied to that animal, i.e., the “side effects” of such therapy.

“Halo” refers to fluoro or chloro.

“Haloalkyl” refers to alkyl as defined above substituted by one or more, for example from one to thirteen, preferably from one to seven, “halo” atoms, as such terms are defined in this Application. Haloalkyl includes monohaloalkyl, dihaloalkyl, trihaloalkyl, perhaloalkyl and the like e.g. chloromethyl, dichloromethyl, difluoromethyl, trifluoromethyl, 2,2,2-trifluoroethyl, perfluoroethyl, 2,2,2-trifluoro-1,1-dichloroethyl, and the like.

“Haloalkylene” means alkylene radical as defined above wherein one to four, preferably one or two hydrogen atoms in the alkylene chain has(have) been replaced by fluorine atom(s).

“Haloalkoxy” refers to a —OR radical where R is haloalkyl group as defined above e.g., trifluoromethoxy, 2,2,2-trifluoroethoxy, difluoromethoxy, and the like.

“Hydroxy” means —OH radical. Unless indicated otherwise, the compounds of the invention containing hydroxy radicals include protected derivatives thereof. Suitable protecting groups for hydroxy moieties include benzyl and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Isomers” mean compounds of Formula (I) having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center that has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula (I) are meant to be encompassed all possible stereoisomers.

“Lower” in reference to a chemical substituent means that it has from 1 to 3 carbon atoms in the substituent chain. For instance, “lower alkyl” can be methyl, ethyl or propyl.

“Optional” or “optionally” or “may be” means that the subsequently described event or circumstance may or may not occur, and that the description includes instances where the event or circumstance occurs and instances in which it does not. For example, the phrase “wherein the aromatic ring in R^{a} is optionally substituted with one or two substituents independently selected from alkyl” means that the aromatic ring may or may not be substituted with alkyl in order to fall within the scope of the invention.

“Pharmaceutically acceptable” means that which is useful in preparing a pharmaceutical composition that is generally safe, and neither biologically nor otherwise undesirable and includes that which is acceptable for veterinary use as well as human pharmaceutical use.

“Pharmaceutically acceptable salts” means salts of compounds of Formula (I) which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxy-ethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of Formula (I). Prodrug means a compound that is convertible in vivo by metabolic means (e.g. by hydrolysis) to a compound of Formula (I). For example, an ester of a compound of Formula (I) containing a hydroxy group may be convertible by hydrolysis in vivo to the parent molecule. Alternatively an ester of a compound of Formula (I) containing a carboxy group may be convertible by hydrolysis in vivo to the parent molecule. Suitable esters of compounds of Formula (I) containing a hydroxy group, are for example acetates, citrates, lactates, tartrates, malonates, oxalates, salicylates, propionates, succinates, fumarates, maleates, methylene-bis-βb-hydroxynaphthoates, gentisates, isethionates, di-p-toluoyltartrates, methylsulphonates, ethanesulphonates, benzenesulphonates, p-toluenesulphonates, cyclohexylsulphamates and quinates. Suitable esters of compounds of Formula (I) containing a carboxy group, are for example those described by Leinweber, F. J. Drug Metab. Res., 1987, 18, page 379. An especially useful class of esters of compounds of Formula (I) containing a hydroxy group, may be formed from acid moieties selected from those described by Bundgaard et al., J. Med. Chem., 1989, 32, pp 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholino-methyl)benzoates, e.g. 3- or 4-(morpholinomethyl)-benzoates, and (4-alkylpiperazin-1-yl)benzoates, e.g. 3- or 4-(4-alkylpiperazin-1-yl)benzoates.

“Protected derivatives” means derivatives of compounds of Formula (I) in which a reactive site or sites are blocked with protecting groups. Protected derivatives of compounds of Formula (I) are useful in the preparation of compounds of Formula (I) or in themselves may be active cathepsin S inhibitors. A comprehensive list of suitable protecting groups can be found in T. W. Greene, Protective Groups in Organic Synthesis, 3rd edition, John Wiley & Sons, Inc. 1999.

### PREFERRED EMBODIMENTS

I. Certain compounds of Formula (I) within the broadest scope set forth in the Summary of the Invention are preferred for use according to the invention. For example:

(A) A preferred group of compounds for use according to the invention is that wherein:

R₁ is cycloalkylalkyl, phenylalkyl, or pyridinylalkyl or aralkyl, wherein any aromatic or aryl ring is optionally substituted with 1 or 2 substituents selected from halo, lower alkyl, CF₃, lower alkoxy, or OCF₃;

R₃ is H or phenyl optionally substituted with 1 or 2 substituents selected from halo, lower alkyl, CF₃, lower alkoxy, or OCF₃.

(B) Another preferred group of compounds for use according to the invention is that wherein:
R₁ is alkyl, cycloalkylalkyl, phenylalkyl, or pyridinylalkyl or aralkyl, wherein any aromatic or aryl ring is optionally substituted with 1 or 2 substituents selected from halo, lower alkyl, CF₃, lower alkoxy, or OCF₃,

R₃ is H or phenyl optionally substituted with 1 or 2 substituents selected from halo, lower alkyl, CF₃, lower alkoxy, or OCF₃

(1) Within the above preferred groups (A) or (B) are compounds wherein

(i) R₃ is H and R₂ is CF₂O-phenyl wherein the phenyl member is optionally substituted with 1 or 2 substituents selected from halo, lower alkyl, CF₃, lower alkoxy, or OCF₃; or

(ii) R₃ is phenyl optionally substituted with 1 or 2 substituents selected from halo, lower alkyl, CF₃, lower alkoxy, or OCF₃ and R₂ is CHF₂, CF₃, or C₂H₅.

(2) Within the above preferred groups (A), A(1), or B(1) are more preferred compounds wherein:

(i) R₁ is cycloalkylmethyl, phenylmethyl, or pyridinylmethyl or phenylmethyl optionally substituted with 1 or 2 substituents selected from halo, lower alkyl, CF₃, lower alkoxy, or OCF₃; or

(ii) R₁ is cyclopropylmethyl.

(3) Within the above preferred groups (A), (B), A(1), B(1), A(2), or B(2) are more preferred compounds wherein

(i) the R₃ phenyl member or the R₂ CF₂O-phenyl member is substituted with 0, 1 or 2 substituents and the R₁ member is substituted with 1 member; or

(ii) the R₃ phenyl member or the R₂ CF₂O-phenyl member is substituted with 1 substituent and the R₁ member is unsubstituted.

(4) Within the above preferred groups (A), (B), A(1), B(1), A(2), B(2), A(3), B(3), A(1)(3), B(1)(3), A(2)(3), B(2)(3), (A)(1)(2)(3), or (B)(1)(2)(3) are more preferred compounds wherein:

(i) the substituents, if present, are selected from halo;

(ii) the substituents, if present, are selected from halo and lower alkyl;

(iii) the substituents, if present, are selected from halo and CF₃; or

(iv) the substituents, if present, are selected from halo, lower alkyl, and trifluoromethyl.

(5) Within the above preferred groups (A)(4), (B)(4), A(1)(4), B(1)(4), A(2)(4), B(2)(4), (A)(1)(2)(4), (B)(1)(2)(4), (A)(3)(4), (B)(3)(4), A(1)(3)(4), B(1)(3)(4), A(2)(3)(4), B(2)(3)(4), (A)(l)(2)(3)(4), (B)(1)(2)(3)(4) are more preferred compounds wherein:

(i) the R₃ is 4-fluorophenyl;

(ii) the R₁ is cyclopropylmethyl;

(iii) the R₃ is 4-fluorophenyl and the R₁ phenylalkyl is 4-fluorophenylmethyl.

### General Synthetic Scheme

Compounds of this invention can be made by the methods known in the art. Compounds of Formula (I), and methods of making them, wherein Y is CF₂ are described in U.S. Patent Application Publication No. US 2008/0293819, published on Nov. 28, 2008 and U.S. Patent Application Publication No. 2008/0214676, published on Sep. 4, 2008, each of which are incorporated herein by reference in its entirety and in particular with respect to such methods. Compounds of Formula I and methods of making them wherein Y is CF₂ or SO₂ and R₂ is CF₂Oaryl are described in PCT Patent Publication No. WO2010/056877, published on May 20, 2010, which is incorporated herein by reference in its entirety and particularly with respect to such compounds and methods. Compounds wherein Y is SO₂ are taught in U.S. Pat. No. 7,547,701, issued on Jun. 16, 2009, and incorporated herein by reference in its entirety and particularly with reference to such compounds and methods. Compounds wherein Y is SO₂ are also taught in U.S. Patent Application Publication No. US2008/0287446, published on Nov. 20, 2008 and incorporated by reference in its entirety and particularly with respect to such compounds and methods of making them.

### Pharmacology and Utility

The compounds of the invention are centrally active inhibitors of cathepsin S which may be administered systemically. In view of their brain penetration abilities and Cathepsin S inhibitory activities, they are particularly useful following systemic administration for treating diseases and conditions of the CNS in which microglial cells mediate or contribute to neuronal inflammation, loss, damage or killing. These diseases include, but are not limited to, neurodegerative diseases or disorders, amyloid diseases, Alzheimer's disease (“AD”), Parkinson's disease (“PD”). Parkinson's disease, mild cognitive dysfunction, post operative cognitive dysfunction, dementia, amyotrophic lateral sclerosis, reduced cerebral perfusion, hypoxia, and stroke. In some embodiments, the compounds are administered to patients in need of treatment to reduce loss of neurons or cerebral function in a condition associated with neuronal inflammation or microglial activation.

The cysteine protease inhibitory activities of the compounds of Formula (I) can be conveniently determined by methods known to those of ordinary skill in the art. Suitable in vitro assays for measuring protease activity and the inhibition thereof by test compounds are known. Typically, the assay measures protease-induced hydrolysis of a peptide-based substrate. Details of suitable assays for measuring protease inhibitory activity are set forth in Biological Example 1.

Suitable in vivo methods for screening the effects of cathepsin S inhibitors on microglial function and neuron loss can be conducted in the intact brains of a triple-transgenic Alzheimer's disease mouse model (3×Tg-AD: PS1M146V knockin, transgenic APPSwe and tauP301L)(see, Oddo, S. et al., Neuron 39, 409-421 (2003)). By crossing these transgenic mice with transgenic mice whose neurons express yellow fluorescent protein (YFP) and, optionally, transgenic mice whose microglia further express green fluorescent protein (GFP) it is possible over a period of 1 month by two-photon in vivo imaging to assess the interaction of neurons and microglia in the brain of quadruple or quintuple transgenic mice much as described in Fuhrmann et al., Nature Neuroscience 13(4):411-413 (2010) which is incorporated herein by reference. In this system, the effects of systemically administered cathepsin S inhibitors according to the invention can be monitored over a period of weeks to months of the two-photon in vivo imaging studies to assess the loss of neurons, and the microglial density and velocity around lost neurons.

Compounds can be evaluated for their efficacy in suppressing microglial immune activation by measuring their potency against LPS-induced expression of iNOS and COX-2 and IL-6 secretion. Microglia and Raw 264.7 cells (a microglia-like cell line) can be pretreated with a test compound at various doses for 1 hr followed by 24 hr treatment of LPS (10 ng/ml). Cell lysates are then subjected to Western blotting for iNOS and COX-2 evaluation. IL-6 secretion can be measured from resulting culture medium by ELISA. β-actin can be used as a control. Active compounds inhibit LPS-induced IL-6 secretion by microglia which is beneficial for retaining hippocampal functions during neuro-inflammation.

Compounds can also be evaluated for their efficacy in suppressing microglia-mediated neurotoxicity. Hippocampal organotypic cultures can be established to serve as an ex vivo model for evaluation of hippocampal functions. Pre-treatment of the cultures with a test compound is followed by addition of LPS (10 ng/mL) for 24 hr. Hippocampal tissues are harvested and subjected to Western blotting for measuring synaptophysin and post synaptic density protein (PSD)95 levels, the indications for neuronal synatic functions. LPS reduces synaptophysin and PSD95 levels in hippocampal organotypic cultures. A compound which reduces this effect of LPS can protect neurons against LPS toxicity by suppressing microglial activation.

In another related aspect, the invention provides methods of treating Parkinson's disease, traumatic brain injury, amyotrophic lateral sclerosis, concussion, hypoxia, poor brain perfusion, or stroke by administering a cathepsin S inhibitor. In some embodiments, the Cathepsin S inhibitors are compounds as disclosed in any of U.S. Patent Application Publication No. US 2008/0293819, published on Nov. 28, 2008; U.S. Patent Application Publication No. US 2008/0214676, published on Sep. 4, 2008, U.S. Patent Application Publication No. US 2009-0270415 published on Oct. 29, 2009; PCT Patent Publication No. WO2010/056877, published on May 20, 2010; U.S. Pat. No. 7,547,701, issued on Jun. 16, 2009; U.S. Patent Application Publication No. US 2008/0287446, published on Nov. 20, 2008; and U.S. Patent Application Publication No. US 2011/0046406 published on Feb. 24, 2011. Each of these patents and patent application publications are incorporated by reference herein in their entireties and particularly with respect to their disclosures of Cathepsin S inhibitory compounds, their activities, and methods of making the compounds.

### Administration and Pharmaceutical Compositions

In general, compounds of Formula (I) will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of Formula (I) may range from about 10 micrograms per kilogram body weight (μg/kg) per day to about 100 milligram per kilogram body weight (mg/kg) per day, typically from about 100 μg/kg/day to about 10 mg/kg/day. Accordingly, in some embodiments, the therapeutically effective amount is from 1 to 100 mg/kg/day. Therefore, a therapeutically effective amount for an 80 kg human patient may range from about 1 mg/day to about 8 g/day, typically from about 1 mg/day to about 800 mg/day. In general, one of ordinary skill in the art, acting in reliance upon personal knowledge and the disclosure of this Application, will be able to ascertain a therapeutically effective amount of a compound of Formula (I) for treating a given disease. In accordance with typical body weights and dosage regimes, in some embodiments, unit dosages may be in an amount from 1 mg to 100 mg, 100 mg to 1 g, or from 1 to 10 g.

The compounds of Formula (I) can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula (I) in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of Formula (I) in the composition may vary widely depending upon the type of formulation, size of a unit dosage, kind of excipients and other factors known to those of skill in the art of pharmaceutical sciences. In general, a composition of a compound of Formula (I) for treating a given disease will comprise from 0.01% w to 90% w, preferably 5% w to 50% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of Formula (I) are described below.

The invention provides pharmaceutical compositions comprising one of the Cathepsin S inhibitors of Formula I. In prophylactic applications, pharmaceutical compositions or medicaments are administered to a patient susceptible to, or otherwise at risk of a neurodegenerative disease or condition (e.g., AD or PD) in an amount sufficient to eliminate or reduce the risk, lessen the severity, or delay the outset of the disease, including biochemical, histologic and/or behavioral symptoms of the neurodegenerative disease, its complications and intermediate pathological phenotypes presenting during development of the disease. In therapeutic applications, compositions or medicants are administered to a patient suspected of, or already suffering from such a neurodegenerative disease in an amount sufficient to cure, or at least partially arrest, the symptoms of the neurodegenerative disease (biochemical, histologic and/or behavioral), including its complications and intermediate pathological phenotypes in development of the neurodegenerative disease. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given.

Aspects of the present invention are further exemplified, but not limited by, the following examples that illustrate the preparation of compound of Formula I, their biological testing for cathepsin S inhibitory activity, and suitable pharmaceutical formulations.

### Synthetic Examples

### Synthesis of Compounds for Use According to the Invention

### Example 1

### Synthesis of N-(1-cyanocyclopropyl)-3-pyridin-3-ylmethanesulfonyl-2(R)-(2,2,2-trifluoro-1(S)-4-fluorophenylethylamino)propionamide

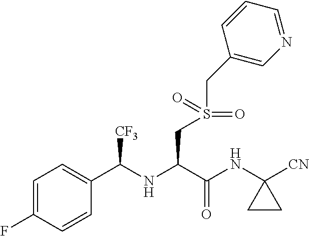

### Step 1

To a solution of 2(R)-[2,2,2-trifluoro-1(S)-(4-fluorophenyl)ethylamino]-3-trityl-sulfanylpropionic acid (539 mg, 1 mmol, 90% de with respect to the purity of the trifluoromethyl group) in CH₂Cl₂ was added trifluoroacetic acid (0.4 mL, 4 mmol) and triethylsilane (0.4 mL, 2 mmol) at 0° C. under nitrogen atmosphere. The reaction mixture was stirred at room temperature for 2 h. The solvent was removed under reduced pressure and the residue was dissolved in 1N NaOH (12 mL). The aqueous layer was washed with hexane and to the basic solution was added dioxane (12 mL), P(CH₂CH₂COOH)₃.HCl (28 mg, 0.1 mmol) and 3-chloromethyl-pyridine (196 mg, 1.2 mmol) and the reaction mixture was stirred at room temperature 2 h. The dioxane was removed under educed pressure and residue was acidified with 6N HCl to pH 5. The product was extracted with ethyl acetate and after drying the organic extracts with MgSO₄, the solvent was removed to give 2(R)-[2,2,2-trifluoro-1(S)-(4-fluorophenyl)ethyl-amino]-3-(pyridin-3-ylmethylsulfanyl)propionic acid which was used in the next step without further purification.

### Step 2

2(R)-[2,2,2-trifluoro-1(S)-(4-fluorophenyl)ethylamino]-3-(pyridin-3-ylmethylsulfanyl)-propionic acid was dissolved in DMF (5 mL) and 1-aminocyclopropanecarbonitrile (142 mg, 1.2 mmol), HATU (456 mg, 1.2 mmol) and NMM (0.44 mL, 4 mmol) were added. After stirring for 2 h at rt, saturated NH₄Cl and ethyl acetate were added and stirring was continued for 20 min. The aqueous layer was extracted with ethyl acetate. The combined organic layers were dried with MgSO₄ and the solvent was removed under the reduced pressure to give N-(1-cyanocyclopropyl)-2(R)-[2,2,2-trifluoro-1(S)-(4-fluorophenyl)ethylamino]-3-(pyridin-3-ylmethylsulfanyl)propionamide as an oil. The crude was used in the next step without further purification.

### Step 3

N-(1-cyanocyclopropyl)-2(R)-[2,2,2-trifluoro-1(S)-(4-fluorophenyl)ethylamino]-3-(pyridin-3-ylmethylsulfanyl)propionamide was dissolved in MeOH (3 mL) and OXONE® (460 mg, 1.5 mmol) in H₂O (3 mL) was added. After stirring at rt for 2 h, the solvent was removed and the residue was extracted with ethyl acetate. The organic layer was dried with MgSO₄ and the solvent was removed under reduced pressure. The title compound was purified by Prep-HPLC.

### Synthetic Example 2

### Synthesis of N-(1-cyanocyclopropyl)-2(R)-[2,2,2-trifluoro-1(S)-(2,4-difluoro-phenyl)ethyl-amino]-3-(cyclopropylmethylsulfonyl)propionamide (compound 43)

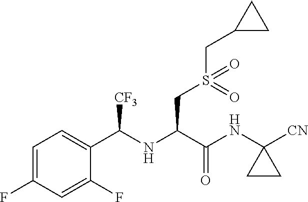

### Step 1

2,4-Difluorobenzaldehyde (1.1 mL, 10.0 mmol) and (trifluoromethyl)trimethylsilane (1.77 mL, 12.0 mmol) were dissolved in THF (25 mL) and then cooled to 0° C. To this, 1M TBAF in THF (76 μL, 76 μmol) was added and the reaction mixture was allowed to warm to room temperature. After 3.25 h, 2.5M HCl (25 mL) was added. The reaction was stirred for 1 h and then extracted with ether. The organic layer was washed with brine and dried with Na₂SO₄. The solvent was removed under the reduced pressure to give 2,2,2-trifluoro-1-(2,4-difluoro-phenyl)ethanol (2.5 g) as a racemic mixture.

### Step 2

2,2,2-Trifluoro-1-(2,4-difluorophenyl)ethanol (1.36 g, 6.4 mmol) was dissolved in dichloromethane (25 mL) and diisopropylethylamine (DIPEA, 5 mL, 28.8 mmol) was added. The resulting solution was cooled to −78° C. and trifluoromethanesulfonic anhydride (1.81 g, 6.4 mmol) was added. After 1 h, the reaction was warmed to −15° C., stirring was continued for 2 h. S-trityl-L-cysteine (2.33 g, 6.4 mmol) was then added and the reaction mixture was stirred overnight. After an aqueous work-up, the organic layer was dried with MgSO₄ then filtered through silica using a combination of ethyl acetate and acetic acid to give a diastereomeric mixture of 2(R)-[1-(2,4-difluorophenyl)-2,2,2-trifluoroethylamino]-3-tritylsulfanylpropionic acid (2.47 g).

### Step 3

2(R)-[1-(2,4-Difluorophenyl)-2,2,2-trifluoroethylamino]-3-tritylsulfanylpropionic acid (2.47 g, 4.4 mmol) was dissolved in a 30% TFA/30% Et₃SiH/40% CH₂Cl₂ v/v/v solution (5 mL). After stirring for 1 h, toluene was added and all solvents were removed under reduced pressure. A basic aqueous work-up was done using 2.7 M NaOH. To the aqueous layer, P(CH₂CH₂COOH)₃ hydrochloride (126 mg, 0.44 mmol) and cyclopropylmethyl bromide (427 μL, 4.4 mmol) were added. After stirring overnight, an acidic aqueous work-up was done. The organic layer was washed with brine and dried with MgSO₄. The solvent was removed to get a diastereomeric mixture of 3-cyclopropylmethylsulfanyl-2(R)-[1-(2,4-difluorophenyl)-2,2,2-trifluoroethylamino]propionic acid (1.25 g).

### Step 4

3-Cyclopropylmethylsulfanyl-2(R)-[1-(2,4-difluorophenyl)-2,2,2-trifluoroethylamino]-propionic acid (1.25 g, 3.4 mmol), HATU (1.29 g, 3.4 mmol), DIPEA (1.48 mL, 8.5 mmol) and 1-amino-cyclopropanecarbonitrile hydrochloride (403 mg, 3.4 mmol) was dissolved in NMP (20 mL). After stirring overnight, Oxone™ (3.14 g, 5.1 mmol) dissolved in water (7.9 mL) was added. After 1 h, more Oxone™ (3.14 g, 5.1 mmol) was added and the reaction mixture was stirred overnight. The product was precipitated from solution by the addition of water. The precipitate was purified by C18 RP-HPLC using an 0.1 mM HCl and acetonitrile system to give a diastereomeric mixture of N-(1-cyanocyclopropyl)-2(R)-[2,2,2-trifluoro-1(R)-(2,4-difluoro-phenyl)ethylamino]-3-(cyclopropylmethylsulfonyl)propionamide and N-(1-cyanocyclopropyl)-2(R)-[2,2,2-trifluoro-1(S)-(2,4-difluorophenyl)ethylamino]-3-(cyclopropylmethylsulfonyl)-propionamide (˜250 mg).

Approximately 50 mg of the diastereomeric mixture was then purified on a Chiralcel OD-H (2 cm×25 cm) HPLC column using a mixture of IPA and hexanes and the diasteriomeric mixture was separated.

N-(1-cyanocyclopropyl)-3-(cyclopropylmethanesulfonyl)-2(R)-(2,2,2-trifluoro-1(S)-2,4-difluorophenylethylamino)propionamide. LC-MS: 466 (M+1), 464 (M−1), 488 (M+23). ¹H-NMR (CDCl₃): 7.50 (s, 1H), 7.43 (q, 1H), 6.99 (m, 1H), 6.90 (m, 1H), 4.65 (q, 1H), 3.71 (dd, J=5.56, 4.95 Hz, 1H), 3.59 (dd, J=14.55, 6.05 Hz, 1H), 3.35 (dd, J=14.56, 4.50 Hz, 1H), 3.03 (d, 2H), 1.18 (m, 4H), 0.77 (m, 2H), 0.45 (m, 2H).

The following compounds were prepared using the appropriated fluorinated benzaldehyde starting materials.

N-(1-cyanocyclopropyl)-3-(cyclopropylmethanesulfonyl)-2(R)-(2,2,2-trifluoro-1(S)-3,5-difluorophenylethylamino)propionamide. LC-MS: 466 (M+H), 488 (M+Na), 464 (M−H), 444 (M−HF).

N-(1-cyanocyclopropyl)-3-(cyclopropylmethanesulfonyl)-2(R)-(2,2,2-trifluoro-1(S)-2,5-difluorophenylethylamino)propionamide. LC-MS: 466 (M+H), 488 (M+Na), 464 (M−H), 444 (M−HF).

N-(1-cyanocyclopropyl)-3-(cyclopropylmethanesulfonyl)-2(R)-(2,2,2-trifluoro-1(S)-2,3-difluorophenylethylamino)propionamide. LC-MS: 466 (M+H), 488 (M+Na), 464 (M−H), 444 (M−HF).

N-(1-cyanocyclopropyl)-3-(cyclopropylmethanesulfonyl)-2(R)-(2,2,2-trifluoro-1(R)-2,5-difluorophenylethylamino)propionamide. LC-MS: 466 (M+H), 488 (M+Na), 464 (M−H), 444 (M−HF).

N-(1-cyanocyclopropyl)-3-(cyclopropylmethanesulfonyl)-2(R)-(2,2,2-trifluoro-1(S)-2,6-difluorophenylethylamino)propionamide. LC-MS: 466 (M+H), 488 (M+Na), 464 (M−H), 444 (M−HF).

N-(1-cyanocyclopropyl)-3-(cyclopropylmethanesulfonyl)-2(R)-(2,2,2-trifluoro-1(R)-2,6-difluorophenylethylamino)propionamide. LC-MS: 466 (M+H), 488 (M+Na), 464 (M−H), 444 (M−HF).

N-(1-cyanocyclopropyl)-3-(cyclopropylmethanesulfonyl)-2(R)-(2,2,2-trifluoro-1(R)-2,3-difluorophenylethylamino)propionamide. LC-MS: 466 (M+H), 488 (M+Na), 464 (M−H), 444 (M−HF).

N-(1-cyanocyclopropyl)-3-(cyclopropylmethanesulfonyl)-2(R)-(2,2,2-trifluoro-1(R)-2,4-difluorophenylethylamino)propionamide. LC-MS: 466 (M+1), 464 (M−1), 488 (M+23). ¹H-NMR (CDCl₃): 7.83 (s, 1H), 7.52 (q, 1H), 7.00 (m, 1H), 6.90 (m, 1H), 4.42 (m, 1H), 3.68 (m, 1H), 3.42 (m, 1H), 3.28 (m, 1H), 2.89 (d, 2H), 1.28 (m, 4H), 0.75 (m, 2H), 0.41 (m, 2H).

N-(1-cyanocyclopropyl)-3-(cyclopropylmethanesulfonyl)-2(R)-(2,2,2-trifluoro-1(S)-2-fluorophenylethylamino)propionamide. LC-MS: 448 (M+H), 470 (M+Na), 446 (M−H), 426 (M−HF).

N-(1-cyanocyclopropyl)-3-(cyclopropylmethanesulfonyl)-2(R)-(2,2,2-trifluoro-1(R)-2-fluorophenylethylamino)propionamide. LC-MS: 448 (M+H), 470 (M+Na), 446 (M−H), 426 (M−HF).

N-(1-cyanocyclopropyl)-3-(1-oxopyridin-2-ylmethanesulfonyl)-2(R)-(2,2,2-trifluoro-1(S)-2-fluorophenylethylamino)propionamide. LC-MS: 501 (M+H), 523 (M+Na), 499 (M−H), 479 (M−HF). This compound was isolated as by-product in the preparation of that gave the corresponding pyridine derivative.

N-(1-cyanocyclopropyl)-3-(1-oxopyridin-2-ylmethanesulfonyl)-2(R)-(2,2,2-trifluoro-1(R)-2-fluorophenylethylamino)propionamide. LC-MS: 501 (M+H), 523 (M+Na), 499 (M−H), 479 (M−HF). This compound was isolated as by-product in the preparation of that gave the corresponding pyridine derivative.

N-(1-cyanocyclopropyl)-3-(pyridin-2-ylmethanesulfonyl)-2(R)-(2,2,2-trifluoro-1(S)-2-fluorophenylethylamino)propionamide. LC-MS: 485 (M+H), 507 (M+Na), 483 (M−H), 463 (M−HF).

N-(1-cyanocyclopropyl)-3-(pyridin-2-ylmethanesulfonyl)-2(R)-(2,2,2-trifluoro-1(R)-2-fluorophenylethylamino)propionamide. LC-MS: 485 (M+H), 507 (M+Na), 483 (M−H), 463 (M−HF).

N-(1-cyanocyclopropyl)-3-(cyclopropylmethanesulfonyl)-2(R)-(2,2,2-trifluoro-1(S)-2,3,4-trifluorophenylethylamino)propionamide. LC-MS: 484 (M+H), 506 (M+Na), 482 (M−H), 462 (M−HF).

N-(1-cyanocyclopropyl)-3-(cyclopropylmethanesulfonyl)-2(R)-(2,2,2-trifluoro-1(R)-2,3,4-trifluorophenylethylamino)propionamide. LC-MS: 484 (M+H), 506 (M+Na), 482 (M−H), 462 (M−HF).

### Synthetic Example 3

### Synthesis of N-(1-cyanocyclopropyl)-3-(cyclopropylmethanesulfonyl)2(R)-[2,2,3,3,3-pentafluoro-1(S)-(4-fluorophenyl)propylamino]propionamide

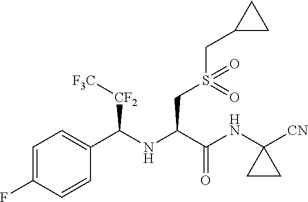

### Step 1

To a solution of 1-bromo-4-fluorobenzene (16.5 mL, 0.15 mol) in anhydrous THF (200 mL) at −78° C., was treated with n-BuLi (60 mL; 150 mmol; 2.5 M in hexane) and the solution was stirred for 1 h. 2,2,3,3,3-pentafluoropropionic acid ethyl ester (14.4 g, 75 mmol; 50 mol %) was added neat added neat. After stirring for 4.5 h at −78° C., ethyl ether and sat. solution of NH₄Cl were added. The layers were separated and the aqueous phase extracted with ethyl ether. The combined organic layers were washed with brine, and dried over sodium sulfate. The solution was concentrated on a rotavap and the residue was purified by distillation to give 2,2,3,3,3-pentafluoro-1-(4-fluorophenyl)propan-1-one (13.1 g; 71%).

### Step 2

To a solution of 2,2,3,3,3-pentafluoro-1-(4-fluorophenyl)propan-1-one (13.0 g, 53 mmol) in a mixture 1:1 of dichloromethane and toluene (160 mL) at room temperature, 1M solution of S-methyl-CBS-oxazaborolidine in toluene (5.3 mL, 5.3 mmol) was added at room temperature. The reaction mixture was cooled at −78° C. and catecholborane (7.62 g, 63 mmol) was added. After stirring for 7 h at −78° C., a 4 M solution of HCl in dioxane (18 mL) was added. After allowing the reaction mixture to warm to room temperature, water (5 mL) was added, stirred for 5 min and 10% solution of sodium metabisulfite (25 mL) was added. The heterogeneous mixture was stirred for 15 min and the solid was separated by filtration. The solution was concentrated on a rotovap to reduce the amount of dichloromethane and then the residue was diluted with hexanes (100 mL). The resulting solution was washed with a 10% solution of sodium metabisulfite (100 mL) and brine (100 mL). After drying over magnesium sulfate, the solution was concentrated and the crude was purified on a silica gel column, using dichloromethane as eluent to give (R)-2,2,3,3,3-pentafluoro-1-(4-fluorophenyl)propan-1-ol as an oil (12.01 g).

### Step 3

A 60% suspension of NaH in oil (2.35 g, 58.8 mmol) was washed several times with hexanes and after suspending it in anhydrous ethyl ether (100 mL), (R)-2,2,3,3,3-pentafluoro-1-(4-fluoro-phenyl)propan-1-ol (12.0 g, 49 mmol) in ether (20 mL) was added slowly at room temperature. After stirring for 15 min, the reaction mixture was cooled at 0° C. and trifluoromethylsulfonyl chloride (12.38 g, 73.7 mmol) was added. The reaction mixture was stirred for 1:30 h at 0° C., then concentrated under reduced pressure and the residue was diluted with hexane (100 mL). The reaction mixture was washed with sat. sol. NaHCO₃, brine and dried over magnesium sulfate. After removing the solvent on a rotovapor, trifluoro-methanesulfonic acid (R)-2,2,3,3,3-pentafluoro-1-(4-fluorophenyl)-propyl ester was obtained as a colorless oil (16.0 g).

### Step 4

To a suspension of L-trityl-cysteine (15.45 g, 42 mmol) and diisopropyl ethyl amine (29.3 mL, 168 mmol) in dichloromethane (350 mL), a solution of trifluoromethanesulfonic acid (R)-2,2,3,3,3-pentafluoro-1-(4-fluorophenyl)-propyl ester (16.0 g, 42 mmol) in dichloromethane (20 mL) was added at room temperature. The reaction mixture was stirred for 20 h and then concentrated. The residue was dissolved in ethyl acetate (300 mL). The organic phase was washed with cold 1N HCl (100 mL), brine and dried over magnesium sulfate. After solvent evaporation, the crude was purified by flash chromatography, using a mixture 1:2 of EA/hexanes to give 2(R)-[2,2,3,3,3-pentafluoro-1-(S)-(4-fluorophenyl)propylamino]-3-tritylsulfanyl-propionic acid as an oil (6.93 g).

### Step 5

To a solution of 2(R)-[2,2,3,3,3-pentafluoro-1-(S)-(4-fluorophenyl)propylamino]-3-tritylsulfanylpropionic acid (6.92 g, 12 mmol) in dichloromethane (10 mL), triethylsilane (3.73 mL, 23.4 mmol) and TFA (3.61 mL, 46.9 mmol) were added at room temperature. The reaction mixture was stirred for 4 h. The solvent and volatiles were evaporated on rotovap, benzene was added to the residue and the mixture was evaporated again to ensure complete removal of excess TFA. The residue was dissolved in 1 N NaOH (50 mL) and extracted with hexanes. To the resulting solution, P(CH₂CH₂CO₂H)₃.HCl (0.343 g, 1.2 mmol) was added, and a 0.2 M stock solution of 2(R)-[2,2,3,3,3-pentafluoro-1-(S)-(4-fluorophenyl)propylamino]-3-mercaptopropionic acid was obtained.

### Step 6

To a 0.2 M stock solution of 2(R)-[2,2,3,3,3-pentafluoro-1-(S)-(4-fluorophenyl)propyl-amino]-3-mercaptopropionic acid in NaOH (10 mL, 2 mmol), cyclopropylmethylbromide (0.270 g, 2 mmol) was added. After stirring the reaction mixture for 5 h at room temperature, 1 M HCl solution was added until pH 2-3. The reaction mixture was extracted with ethyl acetate and the combined organic extracts were washed with brine and dried over sodium sulfate and concentrated to give 3-cyclopropylmethanesulfanyl-2(R)-[2,2,3,3,3-pentafluoro-1-(S)-(4-fluoro-phenyl)propylamino]propionic acid as a foam (0.678 g).

### Step 7

To a solution of 3-cyclopropylmethanesulfanyl-2(R)-[2,2,3,3,3-pentafluoro-1-(S)-(4-fluoro-phenyl)propylamino]-propionic acid (0.670 g, 1.67 mmol) in DMF (3 mL), 1-amino-cyclopropanecarbonitrile hydrogen chloride salt (0.236 g, 3 mmol), HATU (0.760 g, 2 mmol) and diisopropyl ethyl amine (0.87 mL, 5 mmol) were added. After stirring for 4 h, the reaction mixture was diluted with ethyl acetate (20 mL) and then washed with a sat. solution of NaHCO₃ (10 mL), water (10 mL) and brine (10 mL). The crude solution was dried over sodium sufate. After evaporation of the solvent, the crude oil was purified by flash chromatography, using a mixture 1:1 of EA/Hexanes to give N-(1-cyanocyclopropyl)-3-(cyclopropylmethanesulfanyl)-2(R)-(3,3,3,2,2-pentafluoro-1(S)-4-fluorophenylpropylamino)propionamide as a light yellow solid (0.558 g).

### Step 8

To a solution of N-(1-cyanocyclopropyl)-3-(cyclopropylmethanesulfanyl)-2(R)-(3,3,3,2,2-pentafluoro-1(S)-4-fluorophenylpropylamino)propionamide (0.540 g, 1.16 mmol) in N-methylpyrrolidinone (4 mL), a solution of OXONE™ (1.06 g, 1.74 mmol) in water (3.8 mL) was added at room temperature. The heterogeneous mixture was stirred overnight at room temperature. After cooling at 0° C., water (20 mL) was added and mixture was stirred for 15 min. The solid was separated by filtration and washed with fresh water. The crude solid was purified by flash chromatography using a mixture of EA/H as eluent to give title compound as a white solid (0.105 g, 19%). ¹H NMR (DMSO-d₆): δ 8.94 (1H, s), 7.44 (2H, dd), 7.22 (2H, t), 4.53 (1H, m), 3.68 (1H, q), 3.66 (1H, m), 3.30 (2H, m), 3.12 (2H, m), 1.30 (2H, m), 1.00 (1H, m), 0.90 (1H, m), 0.58 (2H, m), 0.47 (1H, m), 0.30 (2H, m). LC/MS, M+1: 498.4, M−1: 496.3.

### Synthetic Example 4

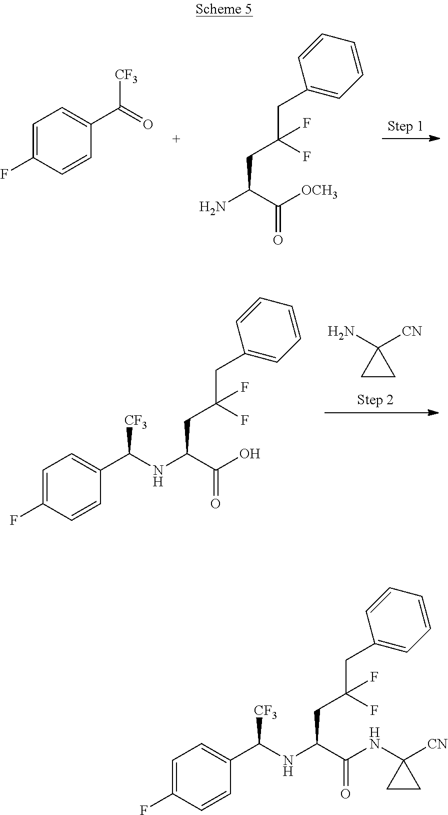

### Scheme 5, Step 1

### (S)-4,4-difluoro-5-phenyl-2-((S)-2,2,2-trifluoro-1-(4-fluorophenyl)ethylamino)pentanoic acid

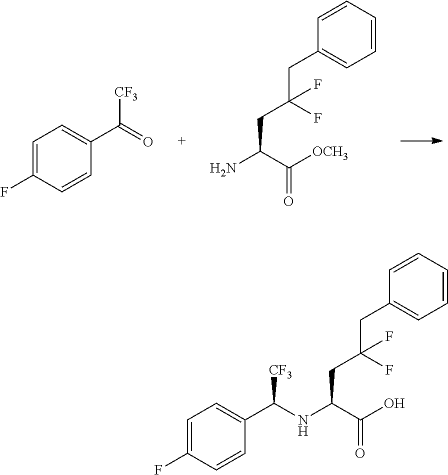

Methyl (S)-methyl 2-amino-4,4-difluoro-5-phenylpentanoate HBr salt (2.44 mmol, 1 eq.) was dissolved in dry methanol. Trifluoromethyl 4-fluorophenyl ketone (2.44 mmol, 1 eq.) and potassium carbonate (4.88 mmol, 2 eq.) were added, and the mixture was heated at 50° C. overnight. To the resulting condensation (imine-formation) reaction product was added, at −30° C., a suspension of Zn(BH₄)₂ (ca. 1.1 eq.) [which was prepared from NaBH₄ (1 eq.) and ZnCl₂ (1M in diethyl ether; 2 eq.)], and the mixture was allowed to warm to RT overnight. The reaction was quenched with 1 N HCl and extracted with ethyl acetate, dried and concentrated to give the crude product, (S)-4,4-difluoro-5-phenyl-2-((S)-2,2,2-trifluoro-1-(4-fluorophenyl)ethylamino)pentanoic acid.

### Scheme 5, Step 2

### (S)—N-(1-cyanocyclopropyl)-4,4-difluoro-5-phenyl-2-((S)-2,2,2-trifluoro-1-(4-fluorophenyl)ethylamino)pentanamide

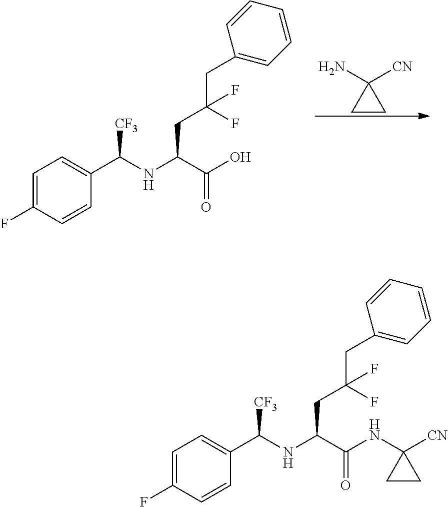

A mixture of the above pentanoic acid (S)-4,4-difluoro-5-phenyl-2-((S)-2,2,2-trifluoro-1-(4-fluorophenyl)ethylamino)pentanoic acid, (1 mmol), 1-aminocyclopropanecarbonitrile hydrochloride (1.2 mmol), HATU (1.2 mmol) and NMM (4.0 mmol), in DMF, was stirred at RT for 2 hr. Saturated ammonium chloride and ethyl acetate were then added, and the reaction was stirred an additional 20 min at RT, after which product was extracted with ethyl acetate, purified with flash column (30-35% ethyl acetate-hexane), and crystallized with DCM-hexane to give (S)—N-(1-cyanocyclopropyl)-4,4-difluoro-5-phenyl-2-((S)-2,2,2-trifluoro-1-(4-fluorophenyl)ethylamino)pentanamide as a white crystal. ¹H-NMR (CDCl₃) δ 8.9 (1H), 7.2-7.5 (9H, m), 4.33 (1H, m), 3.2-3.5 (3H), 2.0-2.6 (2H), 1.6-1.8 (2H), 0.75 (1H), 0.58 (1H). ¹⁹F-NMR (CDCl₃) δ−113 ppm. LC/EIMS (m/z): 470 (M+Na)⁺.

### Synthetic Example 5

### Synthesis of Acid Amides of the Invention

In like manner as in Synthesis Example 4, the following amides are prepared from reaction of 1-aminocyclopropanecarbonitrile hydrochloride with the appropriate carboxylic acid derived from the corresponding difluoroamino acid ester:

(S)—N-(1-cyanocyclopropyl)-4,4-difluoro-4-phenyl-2-((S)-2,2,2-trifluoro-1-(4-fluorophenyl)ethylamino)butanamide.

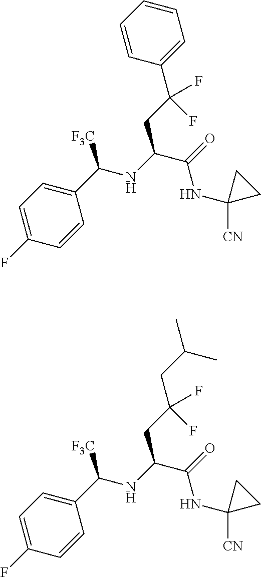

¹H-NMR (CDCl₃) δ 7.4 (2H, m), 7.38-7.42 (2H, m), 6.9 (1H, s), 4.2-4.3 (1H, m), 3.2-3.55 (m), 1.9-2.5 (m), 1.75-1.9 (m), 0.9-1.1 (m). ¹⁹F-NMR (CDCl₃) δ −74.5 (s), −94, −112 (s) ppm. LC/EIMS (m/z): 436 (M+H)⁺

(S)—N-(1-cyanocyclopropyl)-5-cyclopropyl-4,4-difluoro-2-((S)-2,2,2-trifluoro-1-(4-fluorophenyl)ethylamino)pentanamide

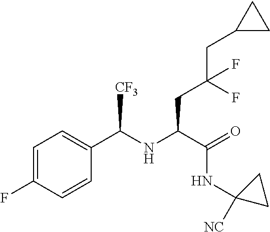

¹H-NMR (CDCl₃) δ 7.45 (1H, s), 7.30-7.35 (2H, m), 7.05-7.15 (2H, m), 4.2-4.3 (1H, m), 3.4-3.5 (1H, m), 2.2-2.6 (3H, m), 1.7-1.9 (2H, m), 1.5-1.6 (m), 1.0-1.3 (2H, m), 0.7-0.9 (1H, m), 0.5-0.6 (2H, m), 0.1-0.2 (2H, m) ¹⁹F-NMR (CDCl₃) δ−74.8 (s), −95.4, −111.5 (s) ppm. LC/EIMS (m/z): 434 (M+H)⁺

(S)—N-(1-cyanocyclopropyl)-4-cyclopropyl-4,4-difluoro-2-((S)-2,2,2-trifluoro-1-(4-fluorophenyl)ethylamino)butanamide

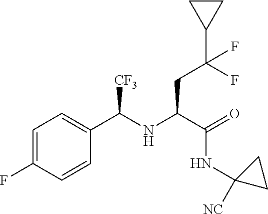

(S)—N-(1-cyanocyclopropyl)-4-cyclohexyl-4,4-difluoro-2-((S)-2,2,2-trifluoro-1-(4-fluorophenyl)ethylamino)butanamide

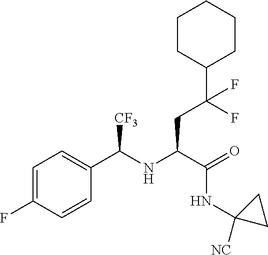

(S)—N-(1-cyanocyclopropyl)-5,5-difluoro-6-phenyl-2-((S)-2,2,2-trifluoro-1-(4-fluorophenyl)ethylamino)hexanamide

### Synthetic Example 6

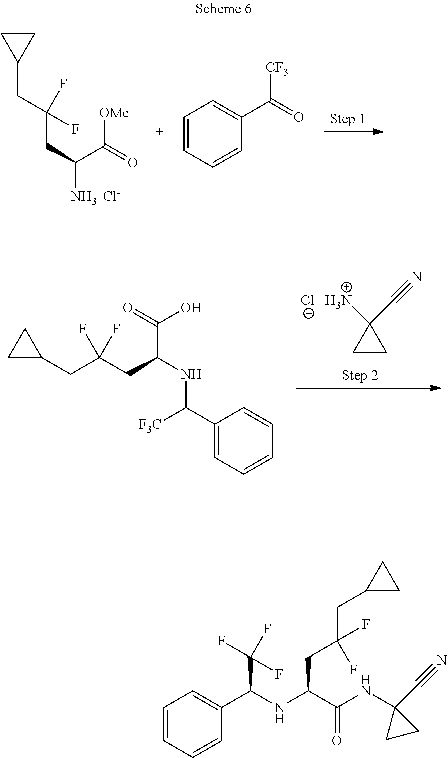

In a manner analogous to the chemistry described in Synthesis Example 4, Scheme 5 additional analogs were synthesized by varying the substitution on the phenyl ring of 2,2,2-trifluoro-1-phenylethanone.

### Scheme 6, Step 1

### (S)-5-cyclopropyl-4,4-difluoro-2-((S)-2,2,2-trifluoro-1-phenylethylamino)pentanoic acid

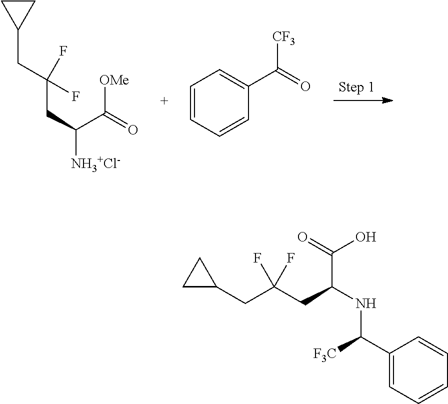

(S)-5-cyclopropyl-4,4-difluoro-1-methoxy-1-oxopentan-2-amine hydrochloride (250 mg, 10 mmol), trifluoroacetophenone (179 mg, 10 mmol), potassium carbonate (426 mg, 30 mmol) was charged in isopropanol (10 mL) under nitrogen. The reaction mixture stirred for 20 h at 60° C. When TLC showed an absence of starting material, is the mixture was filtered under hot and the solids were washed with isopropanol (20 mL) and the filtrates were combined and concentrated under reduced pressure. The residue (400 mg, 10 mmol) was dissolved in acetonitrile (5 mL) and methanol (1 mL) and transferred to a dropping funnel under nitrogen. The zinc borohydride suspension prepared as described above was cooled to −45° C. and treated with the imine solution which was added drop-wise and the reaction was stirred at same temperature for 1 h. Once the staring material was consumed, is the reaction mixture was quenched with 1N HCl solution (10 mL) at 0° C. and then allowed to warm to room temperature. The mixture was extracted with ethyl acetate (3×40 mL) and the combined organic extracts washed with water (50 mL) and brine (50 mL). The organic extracts were evaporated under reduced pressure, and the residue re-dissolved in ethyl acetate (20 mL) and washed with water (20 mL) and brine (50 mL). The solution was dried (MgSO₄) and the solvent evaporated under reduced pressure to give (S)-5-cyclopropyl-4,4-difluoro-2-((S)-2,2,2-trifluoro-1-phenylethylamino)pentanoic acid as a pale oil (265 mg, 71.3%).

Preparation of Zinc Borohydride: Zinc chloride (1.0 g, 7.3 mmol) was suspended in 1,2-DME (10 mL) under nitrogen and stirred for 1 h at room temperature. The resulting white slurry was cooled to ˜5° C. and treated in portions with sodium borohydride (550 mg, 14 mmol). The ice bath was removed and the mixture was stirred at room temperature for 24 h to give a light grey suspension of Zn(BH₄)₂.

### Scheme 6, Step 2

### (S)—N-(1-cyanocyclopropyl)-5-cyclopropyl-4,4-difluoro-2-((S)-2,2,2-trifluoro-1-phenylethylamino)pentanamide

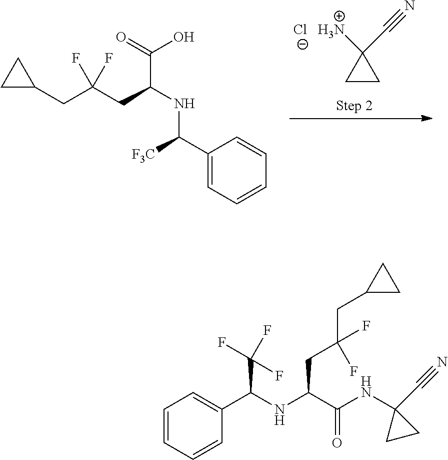

A stirred solution of (S)-5-cyclopropyl-4,4-difluoro-2-(((S)-2,2,2-trifluoro-1-phenylethylamino)pentanoic acid (150 mg, 0.4 mmol) in DMF (15 mL) was treated with HATU (253 mg, 0.5 mmol) and DIPEA (0.3 mL, 1.7 mL mmol). After 15 min, 1-cyanocyclopropane hydrochloride (61 mg, 0.5 mmol) was added and the reaction mixture was stirred under a nitrogen atmosphere for 2 h. Once the reaction was complete, the reaction mixture was quenched with saturated sodium bicarbonate solution, extracted with ethyl acetate & washed with 1 HCl solution and brine, dried over Na₂SO₄, filtered and was concentrated. The crude compound was purified by silica gel column chromatography eluting with 10% ethyl acetate in hexane followed by re-crystallization from DCM and pentane to provide (S)—N-(1-cyanocyclopropyl)-5-cyclopropyl-4,4-difluoro-2-((S)-2,2,2-trifluoro-1-phenylethylamino)pentanamide (65 mg, 37%) as white solid. ¹H-NMR (500 MHz, DMSO-d₆): 8.88 (1H, s, NH), 7.38 (5H, s, Ar—H), 4.23 (1H, m, CHCF₃), 3.39 (1H, m, CH), 3.18 (1H, m, NH), 2.23 (2H, m, CH₂), 1.94 (2H, m, CH₂), 1.32 (2H, m, CH₂), 0.82 (2H, m, CH₂), 0.57 (1H, m, CH), 0.45 (2H, m, CH₂), 0.12 (2H, m, CH₂). ¹⁹F-NMR (500 MHz, CD₃OD): −74.609 (CF₂), −95.308, −95.507 (CF₃). EIMS (m/z): 416 (M+H)¹. HPLC: 97.37% (RT 16.39). Column used Zorbax SB, C8, 250×4.6 mm, 5 u Mobile Phase: ACN (B): 0.1% TFA in water (A). Flow rate: 1.0 mL/Min

### Synthetic Example 7

### Synthesis of Acid Amides of the Invention

In like manner as in Synthesis Example 6, the following amides are prepared from reaction of 1-aminocyclopropanecarbonitrile hydrochloride with the appropriate carboxylic acid derived from the corresponding difluoroamino acid ester:

(S)—N-(1-cyanocyclopropyl)-5-cyclopropyl-4,4-difluoro-2-(((S)-2,2,2-trifluoro-1-(4-methoxyphenyl)ethylamino)pentanamide

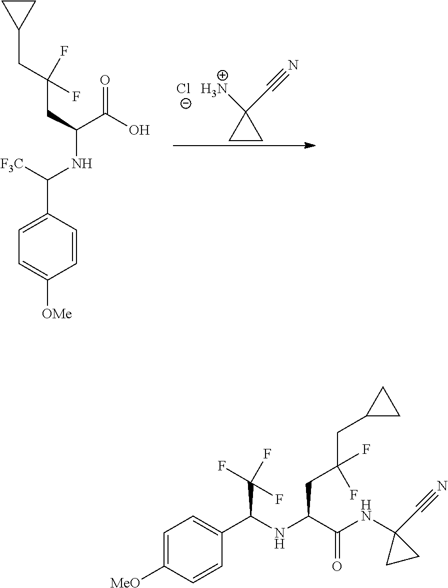

¹H-NMR (500 MHz, CD₃OD): 7.28, 6.93 (4H, A₂B₂, Ar—H), 4.18 (1H, m, CHCF₃), 3.79 (3H, s, OMe), 3.42 (1H, m, CH), 2.33 (2H, m, CH₂), 1.84 (2H, m, CH₂), 1.38 (2H, m, CH₂), 1.01 (1H, m, CH), 0.82 (2H, m, CH₂), 0.53 (2H, m, CH₂), 0.19 (2H, m, CH₂). ¹³C-NMR (500 MHz, CD₃OD): 176.90 (1C, CO), 161.85 (1C, ArC-OMe), 130.95 (2C, ArC), 127.64 (1C, ArC), 125.78 (1C, CF₃), 120.97 (1C, CN), 115.17 (2C, ArC), 97.26 (2C, CF₂), 64.17 (1C, q, CCF₃), 57.49 (1C, OCH₃), 55.79 (1C, CHN), 42.81 (1C, t, CCF₂), 40.50 (1C, t, CCF₂), 21.02 (1C, C), 16.83, 16.65 (2C, CH₂), 5.53 (1C, CCH), 4.60, 4.52 (2C, CH₂) ¹⁹F-NMR (500 MHz, CD₃OD): −74.609, −74.940 (CF₂), −95.308, −95.606 (CF₃). EIMS (m/z): 446 (M+H)¹. HPLC: 98.95% (RT 16.41) Column used Zorbax SB, C8, 250×4.6 mm, 5 u. Mobile Phase: ACN (A): 0.1% TFA in water (B). Flow rate: 1.5 mL/Min

(S)—N-(1-cyanocyclopropyl)-5-cyclopropyl-2-((S)-1-(3,4-difluorophenyl)-2,2,2-trifluoroethylamino)-4,4-difluoropentanamide

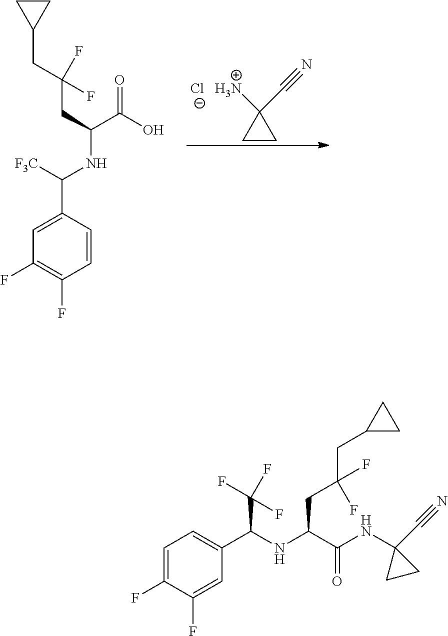

¹H-NMR (500 MHz, CD₃OD): 7.42-7.21 (3H, m, Ar—H), 4.25 (1H, m, CHCF₃), 3.42 (1H, m, CH), 2.37 (2H, m, CH₂), 1.83 (2H, m, CH₂), 1.41 (2H, m, CH₂), 0.99 (2H, m, CH₂), 0.82 (1H, m, CH), 0.57 (2H, m, CH₂), 0.19 (2H, m, CH₂). ¹⁹F-NMR (500 MHz, CD₃OD): −74.808 (CF₂), −95.308, −95.705 (CF₃), −135.646, −135.844 (2×Ar—F). EIMS (m/z): 452 (M+H)¹. HPLC: 94.12% (RT 16.70) Column used zorbax SB, C8, 250×4.6 mm, 5 u. Mobile Phase: ACN (B): 0.1% TFA in water (A) Flow rate: 1.5 mL/Min

(S)—N-(1-cyanocyclopropyl)-5-cyclopropyl-4,4-difluoro-2-((S)-2,2,2-trifluoro-1-(4-(trifluoromethyl)phenyl)ethylamino)pentanamide

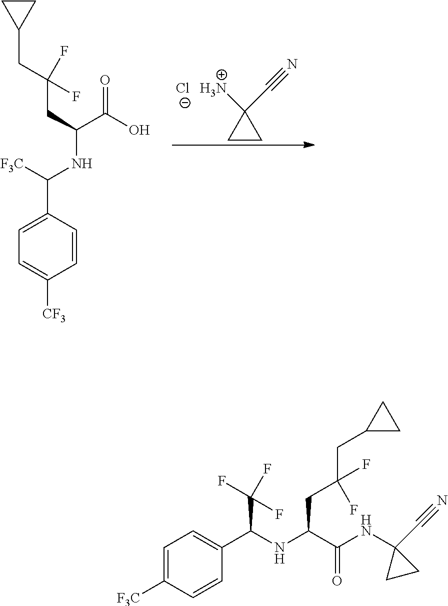

¹H-NMR (500 MHz, CD₃OD): 7.76, 7.62 (4H, A₂B₂, Ar—H), 4.38 (1H, m, CHCF₃), 3.52 (1H, m, CH), 2.38 (2H, m, CH₂), 1.83 (2H, m, CH₂), 1.40 (2H, m, CH₂), 1.02 (1H, m, CH), 0.82 (2H, m, CH₂), 0.53 (2H, m, CH₂), 0.19 (2H, m, CH₂). ¹⁹F-NMR (500 MHz, CD₃OD): EIMS (m/z): 484 (M+H)¹ HPLC: 93.13% (RT 17.14) Column used Zorbax SB, C8, 250×4.6 mm, 5 u. Mobile Phase: ACN (B): 0.1% TFA in water (A). Flow rate: 1.5 mL/Min

### BIOLOGICAL EXAMPLES

### Example 1

### Cathepsin S Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); β-mercaptoethanol, 2.5 mM; and BSA, 0.00%. Human cathepsin S (0.05 pMoles in 25 μL of assay buffer) was added to the dilutions. The assay solutions were mixed for 5-10 seconds on a shaker plate, covered and incubated for 30 min at room temperature. Z-Val-Val-Arg-AMC (4 nMoles in 25 μL of assay buffer containing 10% DMSO) was added to the assay solutions and hydrolysis was followed spectrophotometrically (at λ 460 nm) for 5 min. Apparent inhibition constants (Kᵢ) were calculated from the enzyme progress curves using standard mathematical models.

A number of compounds for use according to the invention were tested by the above-described assay and observed to exhibit cathepsin S inhibitory activity of <or =100 nm.

The following compounds were shown to have a cathepsin S (Kᵢ) of <10 nM:

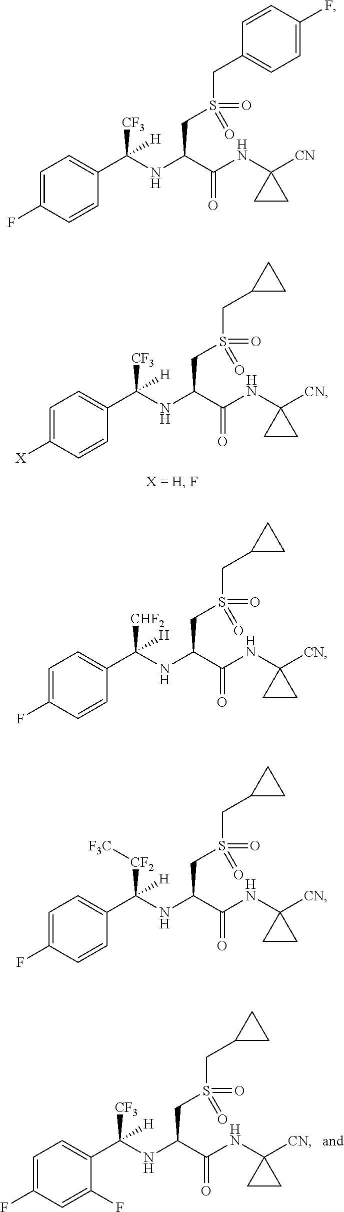

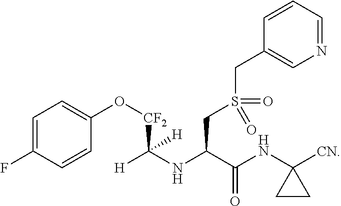

The following compound was shown to have a cathepsin S (Kᵢ) of <10 nM:

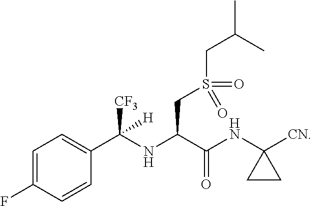

TABLE 1
of Cathepsin S Activity

| | Cathepsin |
|---|---|
| | S |
| Compound | (Kᵢ) [μM] |
| 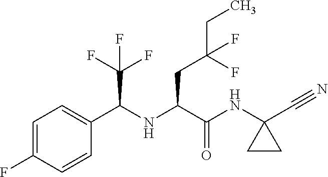 | <0.010 |
| 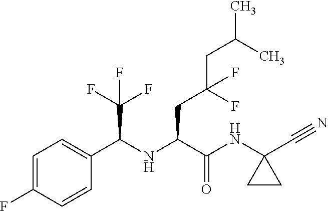 | <0.010 |
| 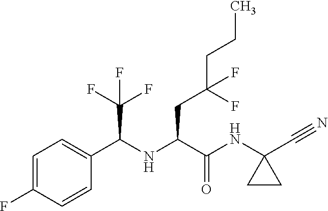 | <0.010 |
| 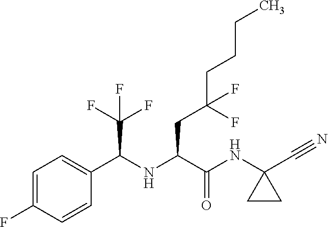 | <0.010 |

TABLE 2
of Cathepsin S Activity

| | | IC50 |
|---|---|---|
| Compound | Enzyme | [nM] |
| 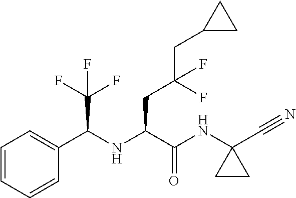 | CatS | <10 |
| 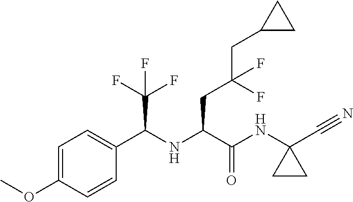 | CatS | <10 |
| 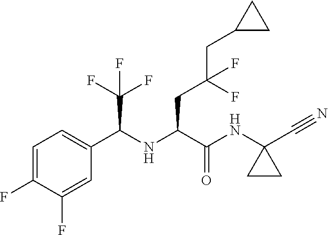 | CatS | <10 |
| 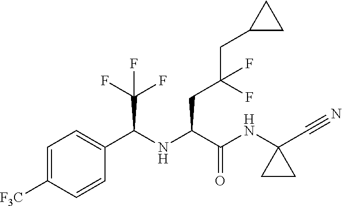 | CatS | <10 |

TABLE 3
of Cathepsin S Activity

| Compound | Cathepsin S (Ki) |
|---|---|
| 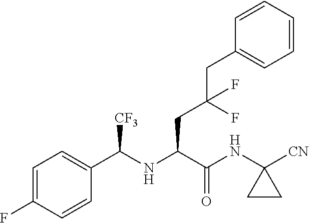 | <20 nM |
| 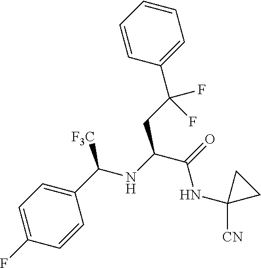 | <20 nM |
| 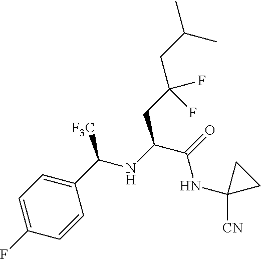 | <20 nM |
| 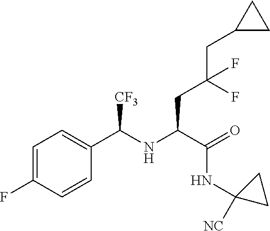 | <20 nM |
| 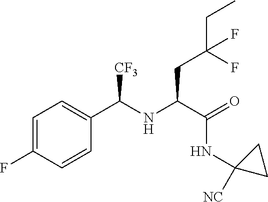 | <20 nM |
| 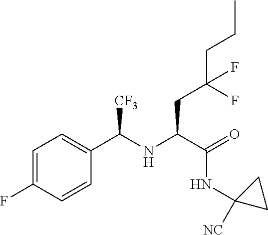 | <20 nM |
| 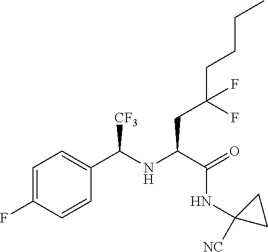 | <20 nM |
| 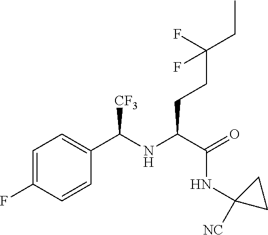 | <20 nM |

### Biological Example 2

### Brain Levels of Cathepsin S Inhibitory Compounds A, B, C, and D

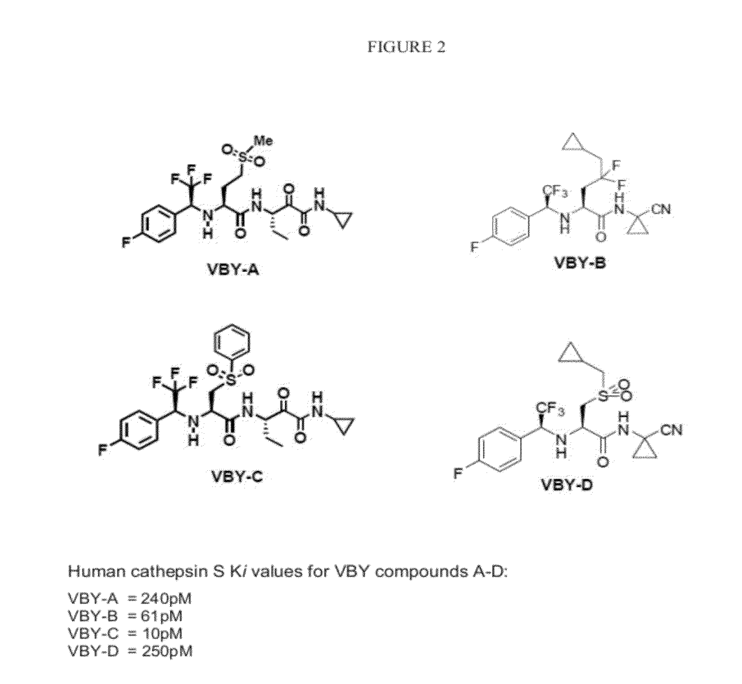

Cathepsin S inhibitors, Compounds A, B, C and D, were each formulated in a methycellulose/Tween 80 suspension and dosed by oral gavage with a single dose in mice or rats. The structures and cathepsin S Kᵢ values for these compounds are shown in FIG. 2. At the 3 hours after dosing, samples were harvested and frozen for pharmacokinetic assessment of drug concentrations in the plasma, brain tissue, and blood-free cerebral spinal fluid. Bioanalytical analysis of these samples was conducted to determine the concentration of each compound in each compartment. For the calculations of drug concentration in brain tissue, it was assumed that the density of wet brain tissue is 1 g/mL. 6 mice were dosed in each group with each compound, and the average plasma, brain, and CSF drug concentrations in all 6 mice was assessed.

The results of the studies are shown in FIG. 1. Based upon the amounts of the compounds measured in rat or mouse brain, the compounds of Formula I (VBY-B and VBY-D) were far superior to the tested ketoamides (VBY-A and VBY-C). This finding was particularly true for the mouse for which brain levels of compound B at 3 hours (2600 ng/g) were far greater than the brain levels observed for VBY-A (14 ng/g) or VBY-C (31 ng/g).

### Pharmaceutical Formulations of Compounds for Use According to the Invention

### Composition Example 1

### Representative Pharmaceutical Formulations Containing a Compound of Formula (I)

### Oral Formulation

| | | |
|---|---|---|
| Compound of Formula (I) | 10-100 | mg |
| Citric Acid Monohydrate | 105 | mg |
| Sodium Hydroxide | 18 | mg |
| Flavoring | | |

| | |
|---|---|
| Water | q.s. to 100 mL |

### Intravenous Formulation

| Compound of Formula (I) | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |

| | |
|---|---|
| Water for Injection | q.s. to 1.0 mL |

### Tablet Formulation

| | |
|---|---|
| Compound of Formula (I) | 1% |
| Microcrystalline Cellulose | 73% |
| Stearic Acid | 25% |
| Colloidal Silica | 1% |

Each patent, patent application, and publication cited herein is incorporated by reference in its entirety for all purposes. The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

What is claimed is:

1. A method for treating microglial-mediated inflammation or neuron loss in the central nervous system, said method comprising systemically administering to a subject in need of said treatment a therapeutically effective amount of a cathepsin S inhibitor of Formula I: 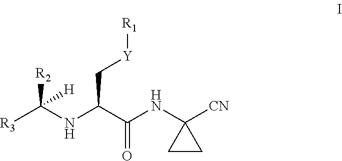
wherein:
Y is SO₂ or CF₂,
R₁ is alkyl, cycloalkyl, alkylcycloalkyl, aryl, aralkyl, or pyridinylalkyl;
R₂ is CHF₂, CF₃, C₂F₅, or CF₂Oaryl;
R₃ is H or aryl, wherein any aryl rings can be optionally substituted with 1 or 2 substituents selected from halo, lower alkyl, CF₃, lower alkoxy, and OCF₃.

2. A method according to claim 1, wherein Y is CF₂.

3. A method according to claim 1, wherein Y is SO₂.

4. A method according to claim 1, wherein the compound is selected from the group consisting of: 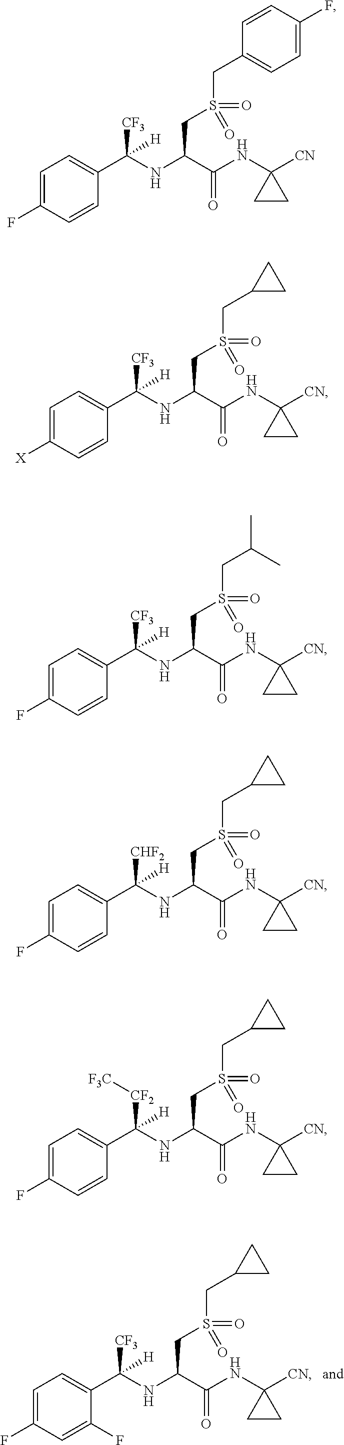 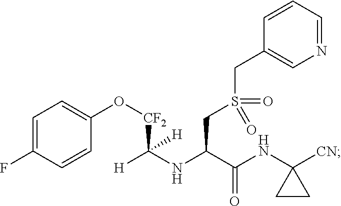
wherein X is H or F.

5. The method of claim 1, wherein Y is CF₂ and R₁ is cycloalkylalkyl.

6. The method of claim 1, wherein the compound is selected from the group consisting of: 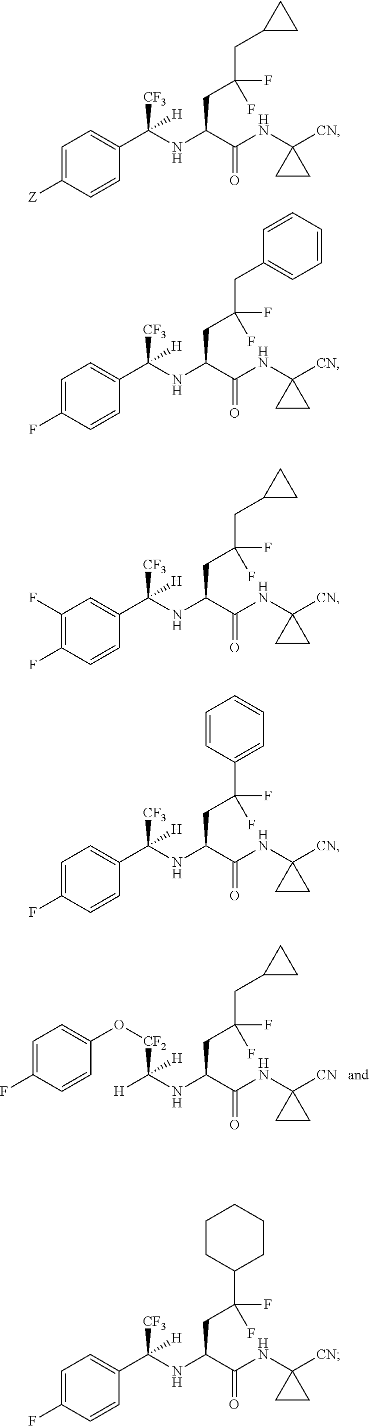
wherein Z is H, F, CF₃, and OCH₃.

7. The method of claim 1, wherein the compound is selected from 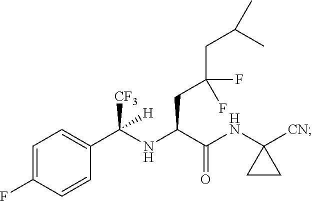
wherein Z is H, F, CF₃, and OCH₃.

8. The method of claim 1, wherein the compound is selected from the group consisting of:

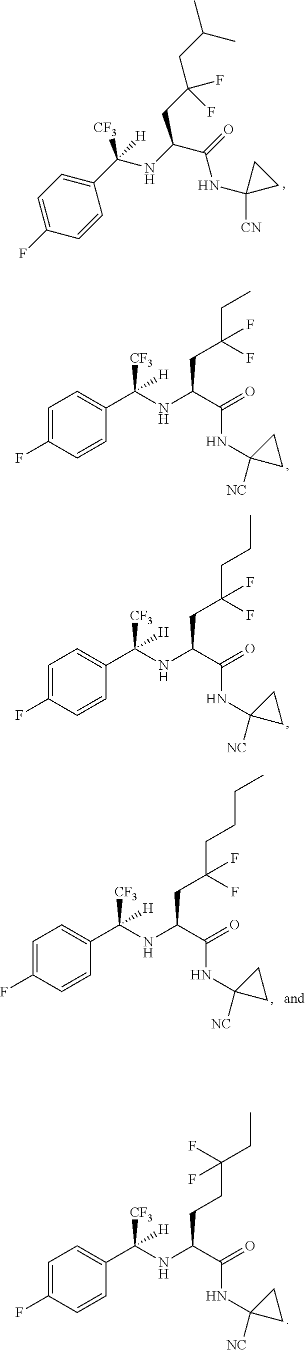

9. The method of claim 1, wherein the R₃ aryl member is substituted with F, CF₃, or OCH₃.

10. The method of claim 1, wherein the R₃ aryl member is a 4-fluorophenyl, 4-methoxy phenyl, or 4-trifluoromethoxyphenyl.

11. The method of claim 1, wherein R₂ is CF₃.

12. The method of claim 1, wherein R₂ is CHF₂.

13. The method of claim 1, wherein R₂ is CF₂F₃.

14. The method of claim 1, wherein the neuronal loss is due to Parkinson's disease, Alzheimer's disease, post operative cognitive dysfunction, or dementia.

15. The method of claim 1, wherein the neuronal loss is due to a neurodegenerative condition.

16. The method of claim 1, wherein the disease is selected from the group consisting of claim 1, wherein the neuronal loss is due to a traumatic brain injury or concussion.

17. The method of claim 1, wherein the neuronal loss is caused by exposure to a neurotoxin, by hypoxia, by stroke or by inadequate brain perfusion.

18. A method of treating a neurodegenerative disease of the CNS, said method comprising administering to a human subject in need of said treatment a therapeutically effective amount of a compound of Formula I: 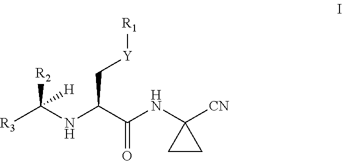
wherein:
Y is SO₂ or CF₂,
R₁ is alkyl, cycloalkyl, alkylcycloalkyl, aryl, aralkyl, or pyridinylalkyl;
R₂ is CHF₂, CF₃, C₂F₅, or CF₂Oaryl;
R₃ is H or aryl, wherein any aryl rings can be optionally substituted with 1 or 2 substituents selected from halo, lower alkyl, CF₃, lower alkoxy, and OCF₃.

19. The method of claim 18, wherein the disease is selected from Parkinson's disease, Alzheimer's disease, post operative cognitive dysfunction, and senile dementia.

20. A method of treating a traumatic brain injury or concussion, said method comprising administering to a subject in need of said treatment a therapeutically effective amount of a compound of Formula I: 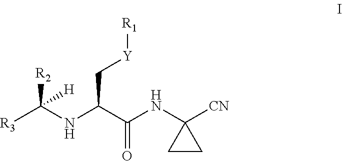
wherein:
Y is SO₂ or CF₂,
R₁ is alkyl, cycloalkyl, alkylcycloalkyl, aryl, aralkyl, or pyridinylalkyl;
R₂ is CHF₂, CF₃, C₂F₅, or CF₂Oaryl;
R₃ is H or aryl, wherein any aryl rings can be optionally substituted with 1 or 2 substituents selected from halo, lower alkyl, CF₃, lower alkoxy, and OCF₃.

21. A method of treating a brain injury, said method comprising systemically administering to a human subject in need of said treatment a therapeutically effective amount of a compound of Formula I: 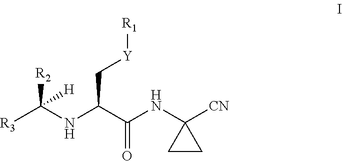
wherein:
Y is SO₂ or CF₂,
R₁ is alkyl, cycloalkyl, alkylcycloalkyl, aryl, aralkyl, or pyridinylalkyl;
R₂ is CHF₂, CF₃, C₂F₅, or CF₂Oaryl;
R₃ is H or aryl, wherein any aryl rings can be optionally substituted with 1 or 2 substituents selected from halo, lower alkyl, CF₃, lower alkoxy, and OCF₃.

22. The method of claim 21, wherein the injury is a neurotoxic injury, traumatic injury, or hypoxic injury.

23. The method of claims 1, 18, and 20-21, wherein the compound is

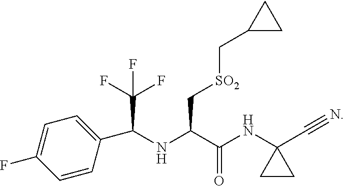

24. The method of claims 1, 18, and 20-21, wherein the compound is

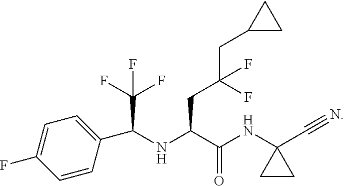

25. The method of claims 1, 18, and 20-21, wherein the compound is

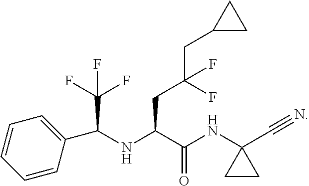

26. The method of claims 1, 18, and 20-21, wherein the compound is [chemical structure]
